(19) United States
(12) Patent Application Publication
(10) Pub. No.: US 2025/0352824 A1
(43) Pub. Date: Nov. 20, 2025

(54) MEASURING RADIATION DOSE

(71) Applicant: LEO CANCER CARE, INC., Middleton, WI (US)

(72) Inventors: Stephen Towe, East Sussex (GB); Mark Strangeman, West Sussex (GB); Michael William Kissick, Middleton, WI (US); Janusz Harasimowicz, Hertfordshire (GB)

(21) Appl. No.: 18/879,430

(22) PCT Filed: Aug. 8, 2023
(86) PCT No.: PCT/US2023/029724

§ 371 Date: Dec. 27, 2024

Related U.S. Application Data
(60) Provisional application No. 63/396,444, filed on Aug. 9, 2022.

(51) Int. Cl. A61N 5/10

(52) U.S. Cl. CPC A61N 5/1075; A61N 2005/1076

(57) ABSTRACT

Provided herein is technology relating to use of radiation for medical purposes and particularly, but not exclusively, to devices, systems, and methods for monitoring, testing, and maintenance of medical radiology equipment as part of a quality assurance program. For example, the technology provides, in part, systems and methods for calculating a tissue phantom ratio (TPR) used to characterize a beam.

This application claims priority to U.S. provisional patent application Ser. No. 63/396,444, filed Aug. 9, 2022, which is incorporated herein by reference in its entirety.

### FIELD

Provided herein is technology relating to use of radiation for medical purposes and particularly, but not exclusively, to devices, systems, and methods for monitoring, testing, and maintenance of medical radiology equipment as part of a quality assurance program.

### BACKGROUND

Medical radiation systems employ radiation sources for imaging and therapeutic purposes, e.g., for radiotherapy. The radiation dose absorbed by a patient is a function of several variables including the radiation beam energy, beam collimation, and distance between the patient and the radiation source.

The radiation dose produced by a medical radiation system can be measured using a “phantom”, typically a “water phantom” comprising a tank filled with water. The water phantom closely approximates the radiation absorption and scattering properties of muscle and other soft biological tissues. The properties of a beam of radiation, after entering the tank and travelling through the water, can be measured using a detector located within the phantom (e.g., within the tank of a water phantom).

The tissue phantom ratio (TPR) is commonly measured to characterize radiation dose provided by a beam. The TPR is defined as the ratio of the dose at a given point in the phantom to the dose at the same point at a fixed reference depth. For example, a common form of TPR is a TPR_{20,10} calculated using radiation measurements recorded at water levels of 10 cm and 20 cm in a water phantom. See, e.g., INTERNATIONAL COMMISSION ON RADIATION UNITS AND MEASUREMENTS, “Determination of Absorbed Dose in a Patient Irradiated by Beams of X or Gamma Rays in Radiotherapy Procedures”, ICRU Rep. 24, ICRU Publications, Bethesda, MD (1976), incorporated herein by reference. See also, “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference.

Current methods for measuring tissue phantom ratio (TPR) require keeping the detector at a fixed position and obtaining radiation measurements at multiple water levels provided between a detector in a water phantom and the radiation source. The water level is usually adjusted manually or automatically through a self-draining mechanism. The draining process may be initially calibrated by a water sensor, which is typically mounted to a moving mechanism of the tank. Further, a water reservoir system may be required to control the water level.

There is a need for new or improved methods for measuring the radiation dose of a medical radiation system.

### SUMMARY

Accordingly, provided herein are embodiments of a technology relating to use of radiation for medical purposes and particularly, but not exclusively, to devices, systems, and methods for monitoring, testing, and maintenance of medical radiology equipment as part of a quality assurance program.

For example, in some embodiments, the technology provides a phantom. In some embodiments, the phantom comprises a tank and the tank comprises water. Water can also be approximated with a solid material (e.g., plastic). See, e.g., Section 4.2.3 and Table 6 in “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference. Accordingly, in some embodiments, the phantom comprises a solid water equivalent. The solid water phantom described herein provides advantages over phantoms comprising liquid water, e.g., when measuring a radiation dose during acceleration or deceleration of the phantom or at any time during which rotation or movement of a liquid water phantom would cause the liquid water level to vary. The technology provided herein relates to embodiments of a phantom comprising liquid water (a “water phantom”) and embodiments of a phantom comprising a solid water equivalent (a “solid phantom”).

In some embodiments, a phantom is a water phantom. In some embodiments, the water phantom comprises a tank comprising a base, a first wall (e.g., radiolucent wall), and a second wall (e.g., radiolucent wall); a detector located within the tank at a first distance from the first wall and located at a second distance from the second wall; and water. In some embodiments, the first wall and/or the second wall comprises poly(methyl methacrylate). In some embodiments, the first wall is at an angle of 90° from the second wall. In some embodiments, the detector has a cylindrical shape. In some embodiments, the detector has a first detection face parallel (e.g., substantially and/or effectively parallel) with the first wall and a second detection face parallel (e.g., substantially and/or effectively parallel) with the second wall. In some embodiments, the first distance is 10 cm and the second distance is 20 cm. Accordingly, in some embodiments, the water phantom finds use in calculating a TPR_{20,10}. In some embodiments, the TPR_{20,10} is a tissue phantom ratio in water at depths of 20 and 10 g/cm², for a field size of 10 cm×10 cm, and a source-chamber distance of 100 cm, which is used as a beam quality index. See, e.g., “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference, and particularly in Section 6.3.2, Table 12, and/or FIG. 6 of this reference.

In some embodiments, the water phantom further comprises a component (e.g., an interface, a mounting component, etc.) structured to attach the water phantom to a patient support assembly. In some embodiments, the detector is located at an axis of rotation of the water phantom. In some embodiments, the water phantom further comprises a movable arm operatively engaged with the detector.

In some embodiments, the phantom is a solid phantom. In some embodiments, the solid phantom comprises a solid water equivalent material and the solid phantom comprises a first external surface and a second external surface; a detector located within the solid phantom at a first distance from the first external surface and located at a second distance from the second external surface. In some embodiments, the solid phantom comprises a hole, and a detector is placed in the hole. In some embodiments, the solid phantom comprises a hole located at a first distance from the first external surface and located at a second distance from the second external surface, and a detector is placed in the hole. In some embodiments, the first external surface is at an angle of 90° from the second external surface. In some embodiments, the detector has a cylindrical shape. In some embodiments, the detector has a first detection face parallel (e.g., substantially and/or effectively parallel) with the first external surface and a second detection face parallel (e.g., substantially and/or effectively parallel) with the second external surface. In some embodiments, the first distance is 10 cm and the second distance is 20 cm. Accordingly, in some embodiments, the solid phantom finds use in calculating a TPR_{20,10}. In some embodiments, the solid phantom further comprises a component (e.g., an interface, a mounting component, etc.) structured to attach the solid phantom to a patient support assembly. In some embodiments, the detector is located at an axis of rotation of the solid phantom. In some embodiments, the solid phantom further comprises a movable arm operatively engaged with the detector.

In some embodiments, technology provides a system for measuring a radiation dose. In some embodiments, systems comprise a phantom (e.g., a water phantom or a solid phantom) comprising a detector, and further comprising an electrometer, a slip ring, and a computer. In some embodiments, the phantom detector is in electric or electronic communication with the electrometer. In some embodiments, the phantom detector and the electrometer are on the same side of the slip ring. That is, embodiments provide that the phantom detector outputs a signal to the electrometer and the signal does not pass through the slip ring between the phantom detector and the electrometer. Accordingly, in some embodiments, the phantom detector and the electrometer are in direct electrical communication. In some embodiments, the phantom detector is in electric or electronic communication with the electrometer through a cable connecting the detector and the electrometer. In some embodiments, the cable connecting the detector and the electrometer is a triaxial cable. In some embodiments, the electrometer is in electric or electronic communication with a microprocessor (e.g., a computer). In some embodiments, the electrometer outputs a signal that is communicated to a microprocessor (e.g., a computer). In some embodiments, the electrometer outputs a signal that is communicated over a slip ring to a microprocessor (e.g., a computer). In some embodiments, the system further comprises an analog-to-digital converter that converts the electrical (e.g., analog) signal produced by the electrometer into a digital signal for communication (e.g., over the slip ring) to the microprocessor.

The technology further provides embodiments of methods. For example, in some embodiments, the technology provides methods for measuring a radiation dose provided by a medical radiation system. In some embodiments, the medical radiation system comprises a radiation source (e.g., a static source) and a patient rotation system adapted to rotate about a rotation axis. In some embodiments, the method comprises locating a phantom (e.g., a water phantom or a solid phantom) on a patient support assembly of the patient rotation system. In some embodiments, the water phantom comprises liquid (e.g., water) and a detector immersed in the liquid. In some embodiments, the liquid is water, an aqueous solution, and/or a composition comprising water. In some embodiments, the solid phantom comprises a solid water equivalent and a detector is located within the solid water equivalent. In some embodiments, the method further comprises moving (e.g., rotating) the phantom relative to a radiation beam generated by the radiation source; detecting, using the detector, the radiation beam; and calculating the radiation dose of the radiation beam. In some embodiments, detecting the radiation beam occurs while the phantom is moving (e.g., rotating). In some embodiments, the radiation beam is detected multiple times (e.g., one time, two times, three times, four times, five times, six times, seven times, eight times, nine times, ten times, or more than ten times) while the phantom is moving (e.g., rotating). In some embodiments, methods comprise moving (e.g., rotating) the phantom, stopping the movement (e.g., rotation) of the phantom, and detecting the radiation while the phantom is stationary. In some embodiments, methods comprise moving (e.g., rotating) the phantom, stopping the movement (e.g., rotation) of the phantom, detecting the radiation while the phantom is stationary, and moving (e.g., rotating) the phantom again. In some embodiments, methods comprise multiple iterations (e.g., one time, two times, three times, four times, five times, six times, seven times, eight times, nine times, ten times, or more than ten times) of moving (e.g., rotating) the phantom, stopping the movement (e.g., rotation) of the phantom, and detecting the radiation while the phantom is stationary.

For example, in some embodiments, the technology provides a method of measuring a radiation dose provided by a medical radiation system comprising a radiation source (e.g., a static source) and a patient rotation system adapted to rotate about a rotation axis. In some embodiments, the method comprises locating a phantom (e.g., a water phantom or a solid phantom) on a patient support assembly of the patient rotation system; moving the phantom relative to a radiation beam generated by the radiation source; detecting, using the detector, the radiation beam; and calculating the radiation dose of the radiation beam. In some embodiments, the phantom is a water phantom comprising a tank containing a liquid and a detector immersed in the liquid (e.g., water, an aqueous solution, and/or a composition comprising water). In some embodiments, the phantom is a solid phantom comprising a solid water equivalent material and a detector located within the solid phantom (e.g., in a hole provided in the solid phantom). In some embodiments, the phantom is located on the patient support assembly such that the rotation axis passes through the phantom. In some embodiments, the phantom is located on the patient support assembly such that a sidewall or external surface of the phantom faces the radiation source, and the radiation beam passes through the sidewall or external surface. In some embodiments, the sidewall or external surface is orthogonal to a central axis of the radiation beam. In some embodiments, the sidewall is transparent to the radiation beam. In some embodiments, the solid phantom comprises a solid water equivalent material that is transparent to the radiation beam.

In some embodiments, moving the phantom (e.g., a water phantom or a solid phantom) comprises rotating the phantom by rotating the patient rotation system about the rotation axis. In some embodiments, moving the phantom comprises translating the phantom by translating the patient support assembly relative to the patient rotation system. In some embodiments, the detector is moveable within the phantom.

In some embodiments, detecting the radiation beam comprises positioning the detector to intercept the radiation beam by moving the phantom (e.g., a water phantom or a solid phantom) and/or moving the detector within the phantom. In some embodiments, detecting the radiation beam comprises detecting the radiation beam at a plurality of locations within the phantom (e.g., using a plurality of detectors and/or by moving a detector to a plurality of locations within the phantom).

In some embodiments, calculating the radiation dose of the radiation beam comprises generating a three-dimensional intensity profile of the radiation beam within the phantom (e.g., a water phantom or a solid phantom). In some embodiments, the detector is located on the rotation axis.

In some embodiments, the calculated radiation dose is a first radiation dose obtained for a first orientation of the phantom (e.g., a water phantom or a solid phantom) and the method further comprises rotating the phantom to a second orientation, different from the first orientation, by rotating the patient rotation system about the rotation axis; detecting, using the detector, the radiation beam for the second orientation; calculating a second radiation dose of the radiation beam for the second orientation; and obtaining a tissue phantom ratio by comparing the second radiation dose to the first radiation dose. In some embodiments, the length of a first propagation path of the radiation beam inside the phantom for the first orientation is different from the length of a second propagation path of the radiation beam inside the phantom for the second orientation. In some embodiments, the length of the first propagation path is X cm; the length of the second propagation path is Y cm; and the tissue phantom ratio is a TPR_{X,Y} measurement.

In some embodiments, X is 1 to 100 cm (e.g., 1, 5, 10, 15, 20, 25, 30, 35, 40, 45, 50, 55, 60, 65, 70, 75, 80, 85, 90, 95, or 100 cm). In some embodiments, Y is 1 to 100 cm (e.g., 1, 5, 10, 15, 20, 25, 30, 35, 40, 45, 50, 55, 60, 65, 70, 75, 80, 85, 90, 95, or 100 cm). In an exemplary embodiment, the length of the first propagation path is 10 cm (e.g., X); the length of the second propagation path is 20 cm (e.g., Y); and the tissue phantom ratio is a TPR_{20,10} measurement.

In some embodiments, a solid phantom is used. In some embodiments, the solid phantom comprises a material that is appropriate for measuring x-rays having a particular energy that is to be tested. In some embodiments, the phantom comprises a first hole at a depth of X cm from an external surface of the phantom. In some embodiments, the phantom comprises a second hole at a depth of Y cm from an external surface of the phantom. In some embodiments, the depth X of the first hole is 1 to 100 cm (e.g., 1, 5, 10, 15, 20, 25, 30, 35, 40, 45, 50, 55, 60, 65, 70, 75, 80, 85, 90, 95, or 100 cm). In some embodiments, the depth Y of the second hole is 1 to 100 cm (e.g., 1, 5, 10, 15, 20, 25, 30, 35, 40, 45, 50, 55, 60, 65, 70, 75, 80, 85, 90, 95, or 100 cm). In some embodiments, the solid phantom comprises a hole (e.g., a single hole) that is at a depth of X cm from a first external surface of the phantom and that is at a depth of Y cm from a second external surface of the phantom. In some embodiments, the depth X of the hole is 1 to 100 cm (e.g., 1, 5, 10, 15, 20, 25, 30, 35, 40, 45, 50, 55, 60, 65, 70, 75, 80, 85, 90, 95, or 100 cm) from a first external surface of the phantom and the depth Y of the hole is 1 to 100 cm (e.g., 1, 5, 10, 15, 20, 25, 30, 35, 40, 45, 50, 55, 60, 65, 70, 75, 80, 85, 90, 95, or 100 cm) from the second external surface of the phantom. In an exemplary embodiment, the solid phantom comprises a first hole at a depth of 10 cm from an external surface of the phantom and/or a second hole at a depth of 20 cm from an external surface of the phantom. In an exemplary embodiment, the solid phantom comprises a single hole at a depth of 10 cm from a first external surface of the phantom and/or at a depth of 20 cm from a second external surface of the phantom.

In some embodiments, the holes are drilled to provide for correcting chamber perturbations to the fluence. In some embodiments, the holes are drilled with corrections accounted for a priori. As described herein, in exemplary embodiments, a solid phantom comprising one or more holes (e.g., each at a depth of from 1 to 100 cm (e.g., 10 cm and/or 20 cm) from a first and/or second external surface) can rotate into position without concern for liquid inertial forces causing wobbles of the device materials.

In some embodiments, the length(s) of the propagation path(s) and/or the depth(s) of the hole(s) may vary (e.g., by approximately ±10% (e.g., +1 to 10% (e.g., +1, 2, 3, 4, 5, 6, 7, 8, 9, or 10%))) from the nominal X and Y values described herein, e.g., to account for the effective point of measurement of the detector. Accordingly, in some embodiments, the technology provides methods comprising placing the phantom at a location relative to the detector that provides one or more effective point(s) of measurement that are at the X and Y distances. In some embodiments, the technology provides systems comprising a phantom located at a location relative to the detector that provides one or more effective point(s) of measurement that are at the X and Y distances.

In some embodiments, the technology uses a detector that provides a substantially symmetrical response for measurements of the phantom in the first orientation and the second orientation. In some embodiments, a correction factor is determined for measurements made using the phantom in the first orientation of the phantom and for measurements made using the phantom in the second orientation.

In some embodiments, a first sidewall or external surface of the phantom (e.g., a water phantom or a solid phantom) facing the radiation source in the first orientation is orthogonal to a central axis of the radiation beam. In some embodiments, a second sidewall or external surface of the phantom facing the radiation source in the second orientation is orthogonal to the central axis of the radiation beam. In some embodiments, the radiation source is an imaging radiation source or a therapeutic radiation source.

In some embodiments, the rotation axis is perpendicular to the radiation beam (e.g., a central axis of the radiation beam). In some embodiments, the rotation axis is a vertical axis.

In some embodiments, the phantom (e.g., a water phantom or a solid phantom) is securely attached to the patient support assembly. In some embodiments, the patient support assembly comprises an interface for attaching the phantom at a fixed position on the patient support assembly. In some embodiments, the phantom is mounted to a seat member of the patient support assembly. In some embodiments, the phantom is mounted to an arm rest of the patient support assembly. In some embodiments, the phantom is located on a horizontal surface of the patient support assembly. In some embodiments, the phantom is disposed horizontally on the patient support assembly such that a central axis of the radiation beam is parallel (e.g., substantially and/or effectively parallel) to a base of the phantom.

Further embodiments of the technology are related to systems. For example, in some embodiments, the technology provides a system comprising a medical radiation system; a phantom (e.g., a water phantom or a solid phantom) comprising a base, a first wall (e.g., radiolucent wall) or first external surface, and a second wall (e.g., radiolucent wall) or second external surface; a detector located within the phantom at a first distance from the first wall or first external surface and located at a second distance from the second wall or second external surface. In some embodiments, the phantom is water phantom comprising a tank (e.g., comprising a base, a first wall, and a second wall) and a liquid (e.g., water, an aqueous solution, and/or a composition comprising water). In some embodiments, the phantom is a solid phantom (e.g., comprising a solid water equivalent material comprising a base, a first external surface, and a second external surface). In some embodiments, the solid phantom comprises a number of holes. In some embodiments, the solid phantom comprises a detector placed within a hole. In some embodiments, the solid phantom comprises a plurality of holes and a plurality of detectors, wherein each of the plurality of detectors is placed in a hole.

In some embodiments, the system further comprises a source (e.g., a static source). In some embodiments, the system further comprises a beam (e.g., a beam produced by the source).

In some embodiments, the system further comprises a patient support assembly. In some embodiments, the patient support assembly comprises an interface structured to accept the phantom. In some embodiments, the patient support assembly is structured to operably engage the phantom. In some embodiments, the patient support assembly is structured to move the phantom. In some embodiments, the patient support assembly is structured to rotate the phantom.

In some embodiments, systems comprise a phantom (e.g., a water phantom or a solid phantom) comprising a detector, and further comprising an electrometer, a slip ring, and a computer. In some embodiments, the detector is in electric or electronic communication with the electrometer. In some embodiments, the detector is in electric or electronic communication with the electrometer through a cable connecting the detector and the electrometer. In some embodiments, the cable connecting the detector and the electrometer is a triaxial cable. In some embodiments, the electrometer is in electric or electronic communication with a microprocessor (e.g., a computer). In some embodiments, the electrometer outputs a signal that is communicated to a microprocessor (e.g., a computer). In some embodiments, the electrometer outputs a signal that is communicated over a slip ring to a microprocessor (e.g., a computer). In some embodiments, the system further comprises an analog-to-digital converter that converts the electrical (e.g., analog) signal produced by the electrometer into a digital signal for communication (e.g., over the slip ring) to the microprocessor.

Thus, in some embodiments, the system is a phantom system comprising a phantom comprising a detector; a slip ring; a microprocessor; and an electrometer in electronic or electric communication with the detector through a cable and in electronic or electric communication with the microprocessor through the slip ring. In some embodiments, the phantom is a water phantom. In some embodiments, the phantom is a solid phantom. In some embodiments, the cable is a triaxial cable. In some embodiments, a computer comprises the electrometer. In some embodiments, the phantom system further comprises an analog-to-digital converter in electric communication with the electrometer. In some embodiments, the phantom system comprises a rotating subsystem comprising the phantom and electrometer. In some embodiments, the phantom system comprises a non-rotating subsystem comprising the microprocessor.

In some embodiments, the technology relates to methods of measuring a radiation dose provided by a medical radiation system comprising a patient rotation system and a radiation source, the patient rotation system being adapted to rotate about a rotation axis. For example, in some embodiments, the method comprises locating a phantom of a phantom system on a patient support assembly of the patient rotation system; moving the phantom relative to a radiation beam generated by the radiation source; detecting, using the detector, the radiation beam; producing, by an electrometer, an electrical signal characterizing the radiation beam; communicating the electrical signal from the electrometer through a slip ring to a microprocessor; and calculating the radiation dose of the radiation beam using the signal. In some embodiments, the phantom is located on the patient support assembly such that the rotation axis passes through the phantom. In some embodiments, moving the phantom comprises rotating the phantom by rotating the patient rotation system about the rotation axis. In some embodiments, detecting the radiation beam comprises detecting the radiation beam at a plurality of locations within the phantom. In some embodiments, calculating the radiation dose of the radiation beam comprises generating a three-dimensional intensity profile of the radiation beam within the phantom. In some embodiments, the detector is located in line with the rotation axis.

In some embodiments, the calculated radiation dose is a first radiation dose obtained for a first orientation of the phantom, and the method further comprises rotating the phantom to a second orientation, different from the first orientation, by rotating the patient rotation system about the rotation axis; detecting, using the detector, the radiation beam for the second orientation; producing, by the electrometer, a second electrical signal characterizing the radiation beam for the second orientation; communicating the second electrical signal from the electrometer through the slip ring to a microprocessor; calculating a second radiation dose of the radiation beam for the second orientation; and obtaining a tissue phantom ratio by comparing the second radiation dose to the first radiation dose. In some embodiments, the length of a first propagation path of the radiation beam inside the phantom for the first orientation is different from the length of a second propagation path of the radiation beam inside the phantom for the second orientation. In some embodiments, the length of the first propagation path is X cm; the length of the second propagation path is Y cm; and the tissue phantom ratio is a TPR_{X,Y} measurement. In some embodiments, X is 1 to 100 cm (e.g., 1, 5, 10, 15, 20, 25, 30, 35, 40, 45, 50, 55, 60, 65, 70, 75, 80, 85, 90, 95, or 100 cm). In some embodiments, Y is 1 to 100 cm (e.g., 1, 5, 10, 15, 20, 25, 30, 35, 40, 45, 50, 55, 60, 65, 70, 75, 80, 85, 90, 95, or 100 cm). In an exemplary embodiment, the length of the first propagation path is 10 cm (e.g., X); the length of the second propagation path is 20 cm (e.g., Y); and the tissue phantom ratio is a TPR_{20,10} measurement.

In some embodiments, the technology relates to methods of measuring one or more radiation dose characteristics (e.g., radiation dose flux, cumulative radiation dose, radiation dose symmetry, radiation dose profile shape, radiation dose penumbra, radiation dose distribution) provided by a medical radiation system comprising a patient rotation system and a radiation source, the patient rotation system being adapted to rotate about a rotation axis. In some embodiments, measuring one or more radiation dose characteristics (e.g., radiation dose flux, cumulative radiation dose, radiation dose symmetry, radiation dose profile shape, radiation dose penumbra, and/or radiation dose distribution) comprises locating a phantom of a phantom system on a patient support assembly of the patient rotation system; moving the phantom relative to a radiation beam generated by the radiation source; detecting, using the detector, the radiation beam; producing, by an electrometer, an electrical signal characterizing the radiation beam; communicating the electrical signal from the electrometer through a slip ring to a microprocessor; and calculating one or more radiation dose characteristics (e.g., radiation dose flux, cumulative radiation dose, radiation dose symmetry, radiation dose profile shape, radiation dose penumbra, radiation dose distribution) of the radiation beam using the signal. In some embodiments, the phantom is located on the patient support assembly such that the rotation axis passes through the phantom. In some embodiments, moving the phantom comprises rotating the phantom by rotating the patient rotation system about the rotation axis. In some embodiments, detecting the radiation beam comprises detecting the radiation beam at a plurality of locations within the phantom. In some embodiments, calculating one or more radiation dose characteristics of the radiation beam comprises generating a three-dimensional intensity profile of the radiation beam within the phantom. In some embodiments, the detector is located in line with the rotation axis.

In some embodiments, the one or more radiation dose characteristics is a first radiation dose characteristic obtained for a first orientation of the phantom, and the method further comprises rotating the phantom to a second orientation, different from the first orientation, by rotating the patient rotation system about the rotation axis; detecting, using the detector, the radiation beam for the second orientation; producing, by the electrometer, a second electrical signal characterizing the radiation beam for the second orientation; communicating the second electrical signal from the electrometer through the slip ring to a microprocessor; calculating a second radiation dose characteristic (e.g., radiation dose flux, cumulative radiation dose, radiation dose symmetry, radiation dose profile shape, radiation dose penumbra, radiation dose distribution) of the radiation beam for the second orientation; and comparing the second radiation dose characteristic to the first radiation dose characteristic. In some embodiments, comparing the second radiation dose characteristic to the first radiation dose characteristic provides a tissue phantom ratio. In some embodiments, the length of the first propagation path is X cm; and the length of the second propagation path is Y cm. In some embodiments, the tissue phantom ratio is a TPR_{X,Y} measurement. In some embodiments, X is 1 to 100 cm (e.g., 1, 5, 10, 15, 20, 25, 30, 35, 40, 45, 50, 55, 60, 65, 70, 75, 80, 85, 90, 95, or 100 cm). In some embodiments, Y is 1 to 100 cm (e.g., 1, 5, 10, 15, 20, 25, 30, 35, 40, 45, 50, 55, 60, 65, 70, 75, 80, 85, 90, 95, or 100 cm). In an exemplary embodiment, the length of the first propagation path is 10 cm (e.g., X); and the length of the second propagation path is 20 cm (e.g., Y). In some embodiments, the tissue phantom ratio is a TPR_{20,10} measurement.

In some embodiments, a first sidewall or external surface of the phantom facing the radiation source in the first orientation is orthogonal to a central axis of the radiation beam. In some embodiments, a second sidewall or external surface of the phantom facing the radiation source in the second orientation is orthogonal to the central axis of the radiation beam. In some embodiments, the radiation source is one of an imaging radiation source or a therapeutical radiation source. In some embodiments, the rotation axis is perpendicular to the radiation beam. In some embodiments, the rotation axis is a vertical axis. In some embodiments, the phantom is securely attached to the patient support assembly. In some embodiments, the patient support assembly comprises an interface for attaching the phantom at a fixed position on the patient support assembly. In some embodiments, the phantom is mounted to a seat member of the patient support assembly. In some embodiments, the phantom is mounted to arm rests of the patient support assembly. In some embodiments, the phantom is located on a horizontal surface of the patient support assembly. In some embodiments, the phantom is disposed horizontally on the patient support assembly such that a central axis of the radiation beam is parallel to a base of the phantom. In some embodiments, the phantom is a water phantom comprising a tank, water, and a detector. In some embodiments, the phantom is a solid phantom comprising a solid water equivalent material and a detector.

In some embodiments, systems described herein further comprise a software component comprising instructions for rotating the phantom. In some embodiments, the system further comprises a software component comprising instructions for activating the source to produce a beam. In some embodiments, the system further comprises a software component comprising instructions for receiving data from the detector and calculating a tissue phantom ratio using the data. In some embodiments, the tissue phantom ratio is a TPR_{20,10}. In some embodiments, the first wall and/or the second wall comprises poly(methyl methacrylate). In some embodiments, the first wall is at an angle of 90° from the second wall. In some embodiments, the first external surface is at an angle of 90° from the second external surface. In some embodiments, the detector has a cylindrical shape. In some embodiments, the detector has a first detection face parallel (e.g., substantially and/or effectively parallel) with the first wall or first external surface; and a second detection face parallel (e.g., substantially and/or effectively parallel) with the second wall or second external surface. In some embodiments, the first distance between the first detection face of the detector and the first wall or first external surface is 10 cm and the second distance between the second detection face of the detector and the second wall or second external surface is 20 cm.

In some embodiments, methods comprise providing and/or using a correction factor to account for mass attenuation and/or differences in density between materials. For example, an acrylic material has a density of 1.18 g/cm³ relative to water having a density of 1.00 g/cm³.

Further, point of measurement for a cylindrical phantom is on a central axis of the phantom and, accordingly, the central axis is placed at the reference depth when measuring dose at an individual point. The effective point of measurement is nearer to the source relative to the point of measurement due to the predominantly forward direction of the secondary electrons. Accordingly, the depth-dose curve is shifted toward the source (e.g., to shallower depth). For cylindrical and spherical chambers this shift is provided by 0.6_{rcav} for photon beams and 0.5_{rcav} for electron beams, where rcav is the radius of the ionization chamber cavity.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A is a schematic drawing showing a perspective view of an example medical radiation system.

FIG. 1B is a schematic drawing showing a top view of a medical radiation system.

FIG. 1C is a schematic drawing showing a top view of a medical radiation system. A patient is positioned in the path of a beam.

FIG. 1D is a schematic drawing showing a top view of a medical radiation system comprising two sources. A patient is positioned in the path of a beam produced by one of the two sources.

FIG. 1E is a schematic drawing showing a top view of a medical radiation system comprising two sources. A patient is positioned in the path of a beam produced by one of the two sources.

FIG. 1F is a schematic drawing showing a top view of a medical radiation system. A water phantom is positioned in the path of a beam.

FIG. 2 is a block diagram of an example method of measuring a radiation dose provided by a medical radiation system.

FIG. 3 is a schematic drawing showing a top view of an example system for conducting a three-dimensional radiation dose scan.

FIG. 4A and FIG. 4B are schematic drawings showing a top view of an example system for measuring a tissue phantom ratio (TPR). FIG. 4A is a schematic drawing of a phantom for conducting the TPR measurement in a first orientation and FIG. 4B is a schematic drawing of the phantom in a second orientation.

FIG. 5A and FIG. 5B are schematic drawings of an embodiment of a phantom system comprising a phantom, an electrometer, and a slip ring.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein are embodiments of a technology relating to use of radiation for medical purposes and particularly, but not exclusively, to devices, systems, and methods for monitoring, testing, and maintenance of medical radiology equipment as part of a quality assurance program.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

As used herein, the term “computed tomography” is abbreviated “CT” and refers both to tomographic and non-tomographic radiography. For instance, the term “CT” refers to numerous forms of CT, including but not limited to x-ray CT, positron emission tomography (PET), single-photon emission computed tomography (SPECT), and photon counting computed tomography. Generally, computed tomography (CT) comprises use of an x-ray source and an x-ray detector panel that revolve around a patient and subsequent reconstruction of images into different planes. In embodiments of CT (e.g., devices, apparatuses, methods, and systems provided for CT) described herein, the x-ray source is a static source and the patient is rotated with respect to the static source. Currents for x-rays used in CT describe the current flow from a cathode to an anode and are typically measured in milliamperes (mA).

As used herein, the term “structured to [verb]” means that the identified element or assembly has a structure that is shaped, sized, disposed, coupled, and/or configured to perform the identified verb. For example, a member that is “structured to move” is movably coupled to another element and includes elements that cause the member to move or the member is otherwise configured to move in response to other elements or assemblies. As such, as used herein, “structured to [verb]” recites structure and not function. Further, as used herein, “structured to [verb]” means that the identified element or assembly is intended to, and is designed to, perform the identified verb.

As used herein, the term “associated” means that the elements are part of the same assembly and/or operate together or act upon/with each other in some manner. For example, an automobile has four tires and four hub caps. While all the elements are coupled as part of the automobile, it is understood that each hubcap is “associated” with a specific tire.

As used herein, the term “coupled” refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components. As used herein, “directly coupled” means that two elements are directly in contact with each other. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the term “removably coupled” or “temporarily coupled” means that one component is coupled with another component in an essentially temporary manner. That is, the two components are coupled in such a way that the joining or separation of the components is easy and does not damage the components. Accordingly, “removably coupled” components is readily uncoupled and recoupled without damage to the components.

As used herein, the term “operatively coupled” means that a number of elements or assemblies, each of which is movable between a first position and a second position, or a first configuration and a second configuration, are coupled so that as the first element moves from one position/configuration to the other, the second element moves between positions/configurations as well. It is noted that a first element is “operatively coupled” to another without the opposite being true.

As used herein, the term “rotatably coupled” refers to two or more components that are coupled in a manner such that at least one of the components is rotatable with respect to the other.

As used herein, the term “translatably coupled” refers to two or more components that are coupled in a manner such that at least one of the components is translatable with respect to the other.

As used herein, the term “temporarily disposed” means that a first element or assembly is resting on a second element or assembly in a manner that allows the first element/assembly to be moved without having to decouple or otherwise manipulate the first element. For example, a book simply resting on a table, e.g., the book is not glued or fastened to the table, is “temporarily disposed” on the table.

As used herein, the term “correspond” indicates that two structural components are sized and shaped to be similar to each other and is coupled with a minimum amount of friction. Thus, an opening which “corresponds” to a member is sized slightly larger than the member so that the member may pass through the opening with a minimum amount of friction. This definition is modified if the two components are to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines, two, or more, “corresponding” surfaces, shapes, or lines have generally the same size, shape, and contours.

As used herein, a “path of travel” or “path,” when used in association with an element that moves, includes the space an element moves through when in motion. As such, any element that moves inherently has a “path of travel” or “path.” As used herein, the statement that two or more parts or components “engage” one another shall mean that the elements exert a force or bias against one another either directly or through one or more intermediate elements or components. Further, as used herein with regard to moving parts, a moving part may “engage” another element during the motion from one position to another and/or may “engage” another element once in the described position. Thus, it is understood that the statements, “when element A moves to element A first position, element A engages element B,” and “when element A is in element A first position, element A engages element B” are equivalent statements and mean that element A either engages element B while moving to element A first position and/or element A engages element B while in element A first position.

As used herein, the term “operatively engage” means “engage and move.” That is, “operatively engage” when used in relation to a first component that is structured to move a movable or rotatable second component means that the first component applies a force sufficient to cause the second component to move. For example, a screwdriver is placed into contact with a screw. When no force is applied to the screwdriver, the screwdriver is merely “coupled” to the screw. If an axial force is applied to the screwdriver, tie screwdriver is pressed against the screw and “engages” the screw. However, when a rotational force is applied to the screwdriver, the screwdriver “operatively engages” the screw and causes the screw to rotate. Further, with electronic components, “operatively engage” means that one component controls another component by a control signal or current.

As used herein, the term “number” shall mean one or an integer greater than one (e.g., a plurality).

As used herein, in the phrase “[x] moves between its first position and second position,” or, “[y] is structured to move [x] between its first position and second position,” “[x]” is the name of an element or assembly. Further, when [x] is an element or assembly that moves between a number of positions, the pronoun “its” means “[x],” i.e., the named element or assembly that precedes the pronoun “its.”

As used herein, a “radial side/surface” for a circular or cylindrical body is a side/surface that extends about, or encircles, the center thereof or a height line passing through the center thereof. As used herein, an “axial side/surface” for a circular or cylindrical body is a side that extends in a plane extending generally perpendicular to a height line passing through the center. That is, generally, for a cylindrical soup can, the “radial side/surface” is the generally circular sidewall and the “axial side(s)/surface(s)” are the top and bottom of the soup can.

As used herein, a “diagnostic” test includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject having a cancer or the likelihood that such a subject will respond favorably to a compound (e.g., a pharmaceutical, e.g., a drug) or other treatment.

As used herein, the term “condition” refers generally to a disease, malady, injury, event, or change in health status.

As used herein, the term “treating” or “treatment” with respect to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, “treatment” comprises exposing a patient or a portion thereof (e.g., a tissue, organ, body part, or other localize region of a patient body) to radiation (e.g., electromagnetic radiation, ionizing radiation).

As used herein, the term “beam” refers to a stream of radiation (e.g., electromagnetic wave and/or or particle radiation). In some embodiments, the beam is produced by a source and is restricted to a small-solid angle. In some embodiments, the beam is collimated. In some embodiments, the beam is generally unidirectional. In some embodiments, the beam is divergent.

As used herein, the term “patient” or “subject” refers to a mammalian animal that is identified and/or selected for imaging and/or treatment with radiation.

Accordingly, in some embodiments, a patient or subject is contacted with a beam of radiation, e.g., a primary beam produced by a radiation source. In some embodiments, the patient or subject is a human. In some embodiments, the patient or subject is a veterinary or farm animal, a domestic animal or pet, or animal used for clinical research. In some embodiments, the subject or patient has cancer and/or the subject or patient has either been recognized as having or at risk of having cancer.

As used herein, the term “treatment volume” or “imaging volume” refers to the volume (e.g., tissue) of a patient that is selected for imaging and/or treatment with radiation. For example, in some embodiments, the “treatment volume” or “imaging volume” comprises a tumor in a cancer patient. As used herein, the term “healthy tissue” refers to the volume (e.g., tissue) of a patient that is not and/or does not comprise the treatment volume. In some embodiments, the imaging volume is larger than the treatment volume and comprises the treatment volume.

As used herein, the term “radiation source” or “source” refers to an apparatus that produces radiation (e.g., ionizing radiation) in the form of photons (e.g., described as particles or waves). In some embodiments, a radiation source is a linear accelerator (“linac”) that produces x-rays or electrons to treat a cancer patient by contacting a tumor with the x-ray or electron beam. In some embodiments, the source produces particles (e.g., photons, electrons, neutrons, hadrons, ions (e.g., protons, carbon ions, other heavy ions)). In some embodiments, the source produces electromagnetic waves (e.g., x-rays and gamma rays having a wavelength in the range of approximately 1 μm to approximately 1 nm). While it is understood that radiation can be described as having both wave-like and particle-like aspects, it is sometimes convenient to refer to radiation in terms of waves and sometimes convenient to refer to radiation in terms of particles. Accordingly, both descriptions are used throughout without limiting the technology and with an understanding that the laws of quantum mechanics provide that every particle or quantum entity is described as either a particle or a wave.

As used herein, the term “radiation dose” refers to an amount of radiation energy deposited per unit mass provided by a radiation beam produced by a source and may also refer to the characteristics of a radiation beam providing the radiation. Thus, as used herein, the term “radiation dose” may be characterized by a number of radiation dose characteristics including but not limited to radiation intensity, radiation spectral intensity, radiation dose flux (e.g., an instantaneous or average rate of radiation intensity as a function of time), cumulative radiation dose (e.g., an integrated amount of radiation provided over a length of time), radiation dose symmetry, radiation dose profile shape, radiation dose penumbra, and/or radiation dose distribution. The term “radiation dose” may be also be characterized in terms of variations in radiation dose characteristics and/or measurement errors associated with measurement of radiation dose characteristics.

As used herein, the term “static source” refers to a source that does not revolve around a patient during use of the source for imaging or therapy. In particular, a “static source” remains fixed with respect to an axis passing through the patient while the patient is being imaged or treated. While the patient may rotate around the axis to produce relative motion between the static source and rotating patient that is equivalent to the relative motion of a source revolving around a static patient, a static source does not move with reference to a third object, frame of reference (e.g., a treatment room in which a patient is positioned), or patient axis of rotation during imaging or treatment, while the patient is rotated with respect to the third object, the frame of reference (e.g., the treatment room in which the patient is positioned), or patient axis of rotation through the patient during imaging or treatment. Thus, a static source is installed on a mobile platform and thus the static source may move with respect to the Earth and fixtures on the Earth as the mobile platform moves to transport the static source. Thus, the term “static source” may refer to a mobile “static source” provided that the mobile “static source” does not revolve around an axis of rotation through the patient during imaging or treatment of the patient. Further, the static source may translate and/or revolve around the patient to position the static source prior to imaging or treatment of the patient or after imaging or treatment of the patient. Thus, the term “static source” may refer to a source that translates or revolves around the patient in non-imaging and non-treatment use, e.g., to position the source relative to the patient when the patient is not being imaged and/or treated. In some embodiments, the “static source” is a photon source and thus is referred to as a “static photon source”. In some embodiments, the “static source” is a particle source and thus is referred to as a “static particle source”.

As used herein, the term “Z” refers to an atomic number (e.g., of an element and/or of a material comprising an element). As used herein, the “Z” of a material refers to the atomic number of the element or elements from which the material is made.

As used herein, the term “effective atomic number” or “Z_{eff}” refers to the effective or average atomic number for a compound or mixture of materials (e.g., an alloy). The Z_{eff} may be determined experimentally or estimated according to calculations described by Murty (1965) “Effective Atomic Numbers of Heterogeneous Materials” Nature 207 (4995): 398-99; Taylor (2008) “The effective atomic number of dosimetric gels” Australasian Physics & Engineering Sciences in Medicine 31 (2): 131-38: Taylor (2009) “Electron Interaction with Gel Dosimeters: Effective Atomic Numbers for Collisional, Radiative and Total Interaction Processes” Radiation Research 171 (1): 123-26; Taylor (2011) “Robust determination of effective atomic numbers for electron interactions with TLD-100 and TLD-100H thermoluminescent dosimeters” Nuclear Instruments and Methods in Physics Research Section B: Beam Interactions with Materials and Atoms 269 (8): 770-73; and Taylor (2012) “Robust calculation of effective atomic numbers: The Auto-Z_{eff} software” Medical Physics 39 (4): 1769-78, each of which is incorporated herein by reference. The Auto-Z_{eff} software described by Taylor is freely available for use in calculating the Z_{eff} of compounds or mixtures of materials.

As used herein, the term “solid water equivalent material” refers to a material having characteristics similar to water with respect to interacting with an x-ray beam and transporting (e.g., propagating) radiation through the material. In some embodiments, a “solid water equivalent material” is a plastic. In some embodiments, the solid water equivalent material has a density of approximately 0.90 to 1.20 g/cm³ (e.g., 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, or 1.20). In some embodiments, the solid water equivalent material has a mean atomic number of 5.20 to 6.70 (e.g., 5.20, 5.21, 5.22, 5.23, 5.24, 5.25, 5.26, 5.27, 5.28, 5.29, 5.30, 5.31, 5.32, 5.33, 5.34, 5.35, 5.36, 5.37, 5.38, 5.39, 5.40, 5.41, 5.42, 5.43, 5.44, 5.45, 5.46, 5.47, 5.48, 5.49, 5.50, 5.51, 5.52, 5.53, 5.54, 5.55, 5.56, 5.57, 5.58, 5.59, 5.60, 5.61, 5.62, 5.63, 5.64, 5.65, 5.66, 5.67, 5.68, 5.69, 5.70, 5.71, 5.72, 5.73, 5.74, 5.75, 5.76, 5.77, 5.78, 5.79, 5.80, 5.81, 5.82, 5.83, 5.84, 5.85, 5.86, 5.87, 5.88, 5.89, 5.90, 5.91, 5.92, 5.93, 5.94, 5.95, 5.96, 5.97, 5.98, 5.99, 6.00, 6.01, 6.02, 6.03, 6.04, 6.05, 6.06, 6.07, 6.08, 6.09, 6.10, 6.11, 6.12, 6.13, 6.14, 6.15, 6.16, 6.17, 6.18, 6.19, 6.20, 6.21, 6.22, 6.23, 6.24, 6.25, 6.26, 6.27, 6.28, 6.29, 6.30, 6.31, 6.32, 6.33, 6.34, 6.35, 6.36, 6.37, 6.38, 6.39, 6.40, 6.41, 6.42, 6.43, 6.44, 6.45, 6.46, 6.47, 6.48, 6.49, 6.50, 6.51, 6.52, 6.53, 6.54, 6.55, 6.56, 6.57, 6.58, 6.59, 6.60, 6.61, 6.62, 6.63, 6.64, 6.65, 6.66, 6.67, 6.68, 6.69, or 6.70). See, e.g., Section 4.2.3 and Table 6 in “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference. Without being limiting, exemplary materials that are solid water equivalent materials are, e.g., polystyrene and poly(methyl methacrylate). Characteristics for “Solid water WT1”, “Solid water RMI-457”, “Plastic water”, and “Virtual water” are provided in Table 6 of “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference. See also “Tissue Substitutes in Radiation Dosimetry and Measurement” (1989) INTERNATIONAL COMMISSION ON RADIATION UNITS AND MEASUREMENTS, Rep. 44, ICRU, Bethesda, Maryland; and Agostinelli (1992) “A new water-equivalent plastic for dosimetry calibration” Med. Phys. 19: 774, each of which is incorporated herein by reference.

As used herein, the term “attenuation coefficient” or “linear attenuation coefficient” refers to a measure of the extent to which the radiant flux of a beam is reduced as it passes through a specific material, e.g., as a result of absorption and/or scattering. A “mass attenuation coefficient” of a material may be used in which the attenuation coefficient is normalized per unit density of the material, thus providing a value that is constant for a given element or compound.

As used herein, the term “radiolucent” refers to a material that does not perturb (e.g., does not substantially and/or does not effectively perturb) a measurement if the material is replaced by another material that is also in the beam (e.g., air or water). For example, air is radiolucent compared to vacuum, and acrylic is radiolucent as a wall for water because if the acrylic were replaced by water the measurements would be the same because the buildup and attenuation from the acrylic wall is not sufficiently different from the water it contains to change the measurement outcome significantly provided a correction for mass attenuation is applied.

As used herein, and when used in reference to communicating data or a signal, “in electronic communication” includes both hardline and wireless forms of communication.

As used herein, “in electric communication” means that a current passes, or can pass, between the identified elements. Being “in electric communication” is further dependent upon an element's position or configuration. For example, in a circuit breaker, a movable contact is “in electric communication” with the fixed contact when the contacts are in a closed position. The same movable contact is not “in electric communication” with the fixed contact when the contacts are in the open position. The term “in direct electric communication” means that two elements are in electric communication with each other without any intervening elements other than a wire, cable, or other conductor connecting the two elements.

The term “computer” as used herein generally includes a plurality of electrical and electronic components that provide power, operational control, and protection to the components and modules within the system. For example, a computer can include, among other things, a processing unit (e.g., a microprocessor, a microcontroller, or other suitable programmable device), a memory, input units, and output units. The processing unit can include, among other things, a control unit, an arithmetic logic unit (“ALC”), and a plurality of registers, and can be implemented using a known computer architecture (e.g., a modified Harvard architecture, a von Neumann architecture, etc.). A “microprocessor” or “processor” refers to one or more microprocessors that can be configured to communicate in a stand-alone and/or a distributed environment, and can be configured to communicate via wired or wireless communications with other processors, where such one or more processor can be configured to operate on one or more processor-controlled devices that can be similar or different devices.

The term “memory” as used herein generally refers to any memory storage of the computer and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices. The processing unit can be connected to the memory and execute software instructions that are capable of being stored in a RAM of the memory (e.g., during execution), a ROM of the memory (e.g., on a generally permanent bases), or another non-transitory computer readable medium such as another memory or a disc. “Memory” can include one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the computer can be configured to retrieve from the memory and execute, among other things, instructions related to the processes and methods described herein.

### Medical Radiation System

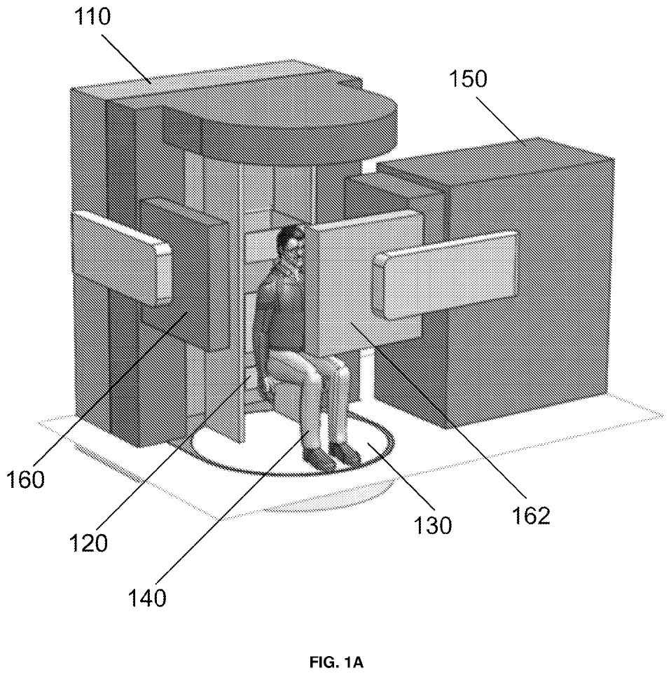

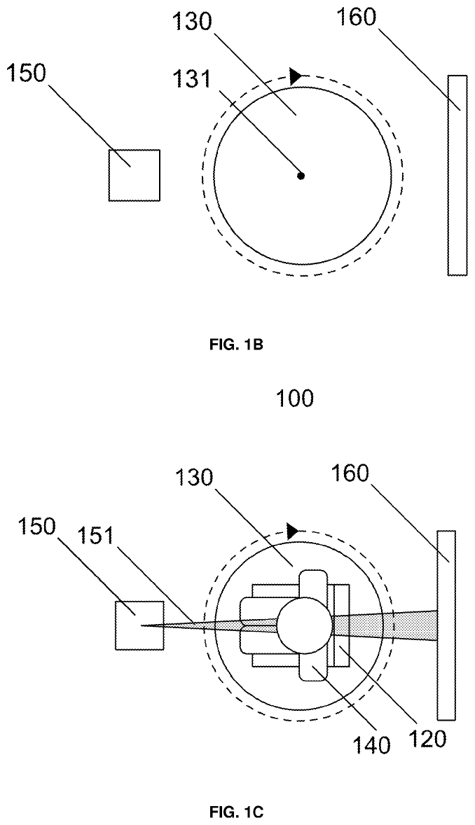

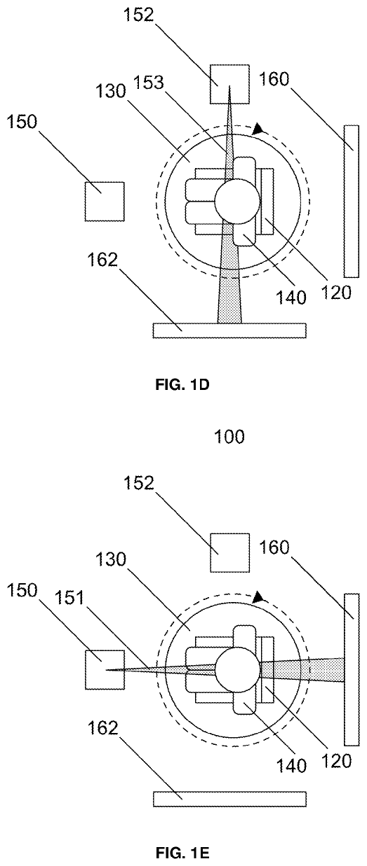

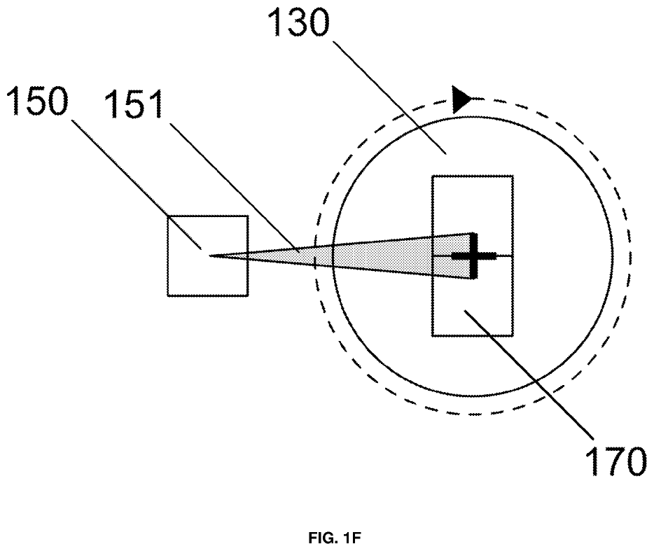

In some embodiments, the technology relates to aligning components of a medical radiation system. In some embodiments, e.g., as shown in FIGS. 1A to 1F, the technology relates to a medical radiation system 100. In some embodiments, medical radiation system 100 comprises a patient rotation system 110 structured to rotate about a rotation axis. In some embodiments, the patient rotation system 110 comprises a patient positioning system (e.g., comprising a patient positioning apparatus or a configurable patient support assembly 120) mounted onto a base 130. Base 130 is structured to rotate about an axis 131 of base 130. In some embodiments, base 130 is structured to rotate about a vertical axis of symmetry of base 130. In some embodiments, the patient positioning system, patient positioning apparatus, and/or configurable patient support 120 is/are as described in U.S. Pat. App. Pub. No. 20200268327 and U.S. Pat. App. Ser. No. 63/237,513, each of which is incorporated herein by reference.

The patient support assembly 120 is structured to support a patient 140 in an upright (e.g., standing, sitting, kneeling, perched) position during radiation treatment or imaging. Accordingly, embodiments provide that the configurable patient support assembly 120 is adjustable to support patient 140 in an upright (e.g., standing, sitting, kneeling, perched) position or any other position in which the torso of patient 140 is in a generally vertical or upright position (e.g., a semi-standing position, a crouching position). In some embodiments, the patient support assembly 120 comprises supporting members such as a seat, a backrest, a headrest, arm rests, shin rests, heel stop, foot brace, and/or a footrest to assist in supporting and/or immobilizing patient 140 in a given position. In some embodiments, e.g., as described in U.S. Pat. App. Ser. No. 63/237,513, which is incorporated herein by reference, the configurable patient support assembly 120 comprises one or more configurable and movable components, e.g., a back rest (e.g., a configurable and movable back rest), a head rest (e.g., a configurable and movable head rest), an arm rest (e.g., a configurable and movable arm rest), a seat member (e.g., a configurable and movable seat member), a shin rest (e.g., a configurable and movable shin rest), a heel stop (e.g., a configurable and movable heel stop), and/or a foot brace (e.g., a configurable and movable foot brace). In some embodiments, one or more configurable and movable components of the configurable patient support comprises one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat member (e.g., a seat member operatively engaged with a seat member motor), a motorized shin rest (e.g., a shin rest operatively engaged with a shin rest motor), a motorized heel stop (e.g., a heel stop operatively engaged with a heel stop motor), and/or a motorized foot brace (e.g., a foot brace operatively engaged with a foot brace motor). In some embodiments, the back rest motor is structured to move (e.g., translate and/or rotate) the back rest, the head rest motor is structured to move (e.g., translate and/or rotate) the head rest, the arm rest motor is structured to move (e.g., translate and/or rotate) the arm rest, the seat member motor is structured to move (e.g., translate and/or rotate) the seam member, the shin rest motor is structured to move (e.g., translate and/or rotate) the shin rest, the heel stop motor is structured to move (e.g., translate and/or rotate) the heel stop, and/or the foot brace motor is structured to move (e.g., translate and/or rotate) the foot brace. In some embodiments, the technology provides a configurable patient support 120 that is configured in a static configuration. In some embodiments, the technology provides a configurable patient support 120 that is configured in a dynamic configuration (e.g., a configuration that moves to assist patient movement, e.g., for patient ingress and/or for patient egress). See U.S. Pat. App. Ser. No. 63/237,513, which is incorporated herein by reference.

In some embodiments, the patient support assembly 120 is operatively coupled to base 130 such that the patient support assembly 120 rotates together with base 130 (e.g., around axis 131). However, patient support assembly 120 may be adjustably mounted to base 130 for adjusting of a position and/or an orientation of patient support assembly 120 relative to base 130. In some embodiments, patient support assembly 120 is configured for movement with six degrees of freedom, permitting translation in three perpendicular axes (e.g., two axes in a horizontal plane and a vertical axis) and rotation about three perpendicular axes (e.g., yaw, pitch, and roll). See U.S. Pat. App. Ser. No. 63/237,513, which is incorporated herein by reference. In some embodiments, patient support assembly 120 is moveable with fewer than six degrees of freedom.

In some embodiments, the medical radiation system 100 comprises a first radiation source 150 structured to generate a beam 151 of electromagnetic radiation. In some embodiments, the first radiation source 150 is a kilovoltage (kV) or a megavoltage (MV) x-ray radiation source. First radiation source 150 may be a therapeutic radiation source or an imaging radiation source. In some embodiments, the medical radiation system further comprises a second radiation source 152 structured to generate a second beam 153 of electromagnetic radiation. Thus, in some embodiments, medical radiation system 100 comprises two radiation sources, e.g., a first radiation source that is a therapeutic radiation source and a second radiation source that is an imaging radiation source. In some embodiments, radiation source 150 is a static source, e.g., a source that is not moveable during normal operation (e.g., during radiotherapy). Accordingly, the radiation source 150 may translate, revolve, and/or rotate during a calibration or alignment procedure. In some embodiments, first radiation source 150 is a static source and/or second radiation source 152 is a static source. Accordingly, the first radiation source 150 and/or the second radiation source 152 may translate, revolve, and/or rotate during a calibration or alignment procedure.

Furthermore, embodiments provide that the radiation beam 151 from the first radiation source 150 is perpendicular to the axis of rotation 131 of base 130 (e.g., following an alignment procedure) and/or that the radiation beam 153 from the second radiation source 152 is perpendicular to the axis 131 of rotation of base 130 (e.g., following an alignment procedure). In some embodiments, the radiation source 150 is oriented such that the radiation beam 151 intersects the axis of rotation 131. In some embodiments, an isocenter of the radiation beam intersects the axis of rotation 131.

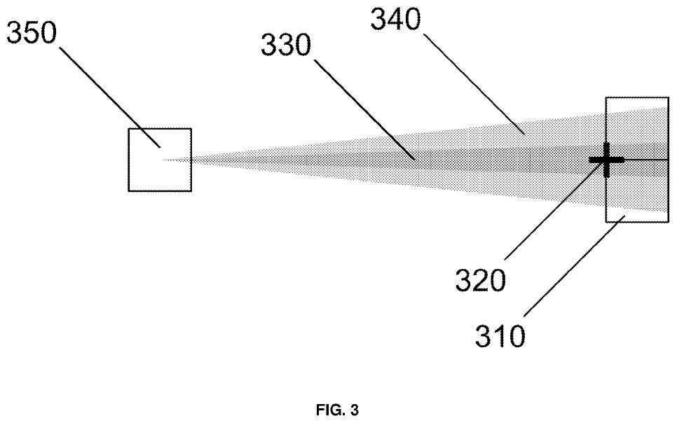

In some embodiments, radiation source 150 is structured to direct a radiation beam 151 in the direction of patient support assembly 120. Accordingly, when a patient 140 is positioned on the patient support assembly 120, radiation source 150 is structured to direct a radiation beam 151 in the direction of patient 140. In some embodiments, the medical radiation system 100 comprises a detector 160 (e.g., a detection panel) provided opposite the radiation source 150 to detect the radiation beam 151 that traverses patient 140. In some embodiments, detector 160 is an imaging device that produces a signal and/or data for generating an image produced by the radiation beam 151. In some embodiments, an additional (e.g., second) detector 162 is associated with the second radiation source 152 of medical radiation system 100. In some embodiments, e.g., as described further herein, a phantom 170 (e.g., a water phantom comprising a tank, liquid, and a detector or a solid phantom comprising a solid water equivalent material (e.g., as shown in FIGS. 3-5)) is placed between the radiation source 150 and the detector 160 (e.g., a detection panel). In some embodiments, e.g., as described further herein, a phantom 170 (e.g., a water phantom comprising a tank, liquid, and a detector or a solid phantom comprising a solid water equivalent material (e.g., as shown in FIGS. 3-5)) is placed between the second radiation source 152 and the second detector 162 (e.g., a detection panel). One of ordinary skill in the art will understand that medical radiation systems comprise a detector and the phantom described herein comprises a detector and that these two detectors are different components of the technology. The detector of the medical radiation system (e.g., detector 160) is placed opposite a source (e.g., radiation source 150), e.g., such that the detector and source of the medical radiation system are located on opposite sides of a patient positioned for imaging and/or treatment with the medical radiation system; and the detector of the phantom is present within the tank of the water phantom or within the solid phantom (e.g., in a hole of the solid phantom), e.g., such that the detector of the phantom may be placed at or near a location of the medical radiation system where a patient would otherwise be located (e.g., on the patient support assembly).

### Phantom

The technology relates to a phantom for use in quality assurance validation of a medical radiation system. In some embodiments, the phantom is a water phantom. In some embodiments, the phantom is a solid phantom. In some embodiments, a water phantom comprises a tank, the tank comprises water, and a detector is placed within the water. Water can also be approximated with a solid material (e.g., plastic). See, e.g., Section 4.2.3 and Table 6 in “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference. Accordingly, in some embodiments, the phantom is a solid phantom comprising a solid water equivalent material. In some embodiments, the solid water equivalent material is specific for a particular beam quality or x-ray energy to be tested.

In some embodiments, the solid phantom described herein provides advantages over phantoms comprising a liquid (e.g., liquid water), e.g., when measuring a radiation dose during acceleration or deceleration of the phantom or at any time during which rotation or movement of a liquid (e.g., liquid water) phantom would cause the liquid (e.g., liquid water) level to vary. Thus, in some embodiments, a solid phantom can be rotated to any measurement angle without the need to stabilize the phantom material prior to performing a measurement.

The technology provided herein relates to embodiments of a phantom comprising liquid water (a “water phantom”) and embodiments of a phantom comprising a solid water equivalent (a “solid phantom”).

In some embodiments, the technology provides a water phantom. In some embodiments, the water phantom comprises a tank and a liquid held within the volume of the tank. In some embodiments, the liquid is water, a composition comprising water, and/or an aqueous solution. In some embodiments, the tank comprises a base (e.g., a surface of the tank that supports the weight of the liquid contained in the tank (e.g., opposing the force of gravity acting on the liquid (e.g., water))) and one or more sidewalls defining a volume for containing a liquid (e.g., water, a composition comprising water, and/or an aqueous solution). In some embodiments, the tank has a polygon (e.g., quadrilateral (e.g., rectangle))) shape when viewed from the top. However, the technology is not limited to tanks having polygon, quadrilateral, and/or rectangle shapes when viewed from the top. Accordingly, embodiments provide tanks having other shapes when viewed from the top, e.g., an “L” shape, a cross shape, a circular shape, etc.

In some embodiments, the sidewalls of the tank comprise a material that is transparent and/or radiolucent to x-ray radiation. In some embodiments, the sidewalls of the tank comprise a material with a low attenuation coefficient (e.g., a radiotransparent or “radiolucent” material), e.g., a low attenuation coefficient relative to other materials in the beam path. In some embodiments, the sidewalls of the tank transmit radiation similarly to water so that the measurement is not perturbed by the radiation passing through the sidewalls. In some embodiments, the sidewalls of the tank comprise a homogenous material with a low attenuation coefficient or having an attenuation coefficient that is similar to water or the liquid contained within the tank (e.g., a radiotransparent or “radiolucent” material). In some embodiments, the material having a low attenuation coefficient is, e.g., poly(methyl methacrylate), which is also known as acrylic glass and sold commercially under the name PERSPEX.

In some embodiments, the tank comprises a detector. In some embodiments, the detector is fully immersed in the liquid of the tank. In some embodiments, the detector is partially immersed in the liquid of the tank. In some embodiments, the detector is structured to measure the energy, intensity, and/or dose of a radiation beam. In some embodiments, the detector is structured to measure one or more other characteristics of the radiation beam (e.g., shape, spectrum, wavelength, flux, photon count, etc.) In some embodiments, the detector comprises an ion chamber.

In some embodiments, the detector is moveable within the tank. In some embodiments, the detector is mounted to an adjustable arm that is fixed to the tank, such that the location and/or orientation of the detector may be adjusted using the adjustable arm. In some embodiments, the tank comprises more than one detector (e.g., in some embodiments, the tank comprises 2, 3, 4, 5, 6, 7, 8, or more detectors). In some embodiments, the tank comprises two detectors that are oriented at right angles to each other, e.g., so that a radiation beam is detected by both detectors as the tank is rotated by 90°. In some embodiments, the tank comprises two detectors that are oriented at an 180° angle to each other, e.g., so that a radiation beam is detected by both detectors as the tank is rotated by 180°. In some embodiments, a detector is removable from the solid phantom, e.g., for calibration, replacement, maintenance, and/or repair.

In some embodiments, the tank comprises a single cylindrical detector that is oriented in a vertical direction along the rotation axis of the tank, e.g., so as to present a detection surface at a constant distance from the source (e.g., at a fixed location) along the axis of the radiation beam for all angles of rotation of the tank. In this way, the distance between the radiation source and the detector remains constant as the tank is rotated.

In some embodiments, the detector is movable within the tank and the tank defines a measurement area (or a measurement volume), e.g., because beam characteristics may be measured at one or more locations within the tank.

In some embodiments, the technology provides a solid phantom. In some embodiments, the solid phantom comprises a solid water equivalent material. In some embodiments, the solid water equivalent material is a material described in “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference, and particularly in Section 4.2.3 and/or Table 6 of this reference.

In some embodiments, the solid phantom comprises a number of holes. In some embodiments, the solid phantom comprises 1 to 10 holes (e.g., 1, 2, 3, 4, 5, 6, 7, 8, 9, or 10 holes). In some embodiments, holes are precisely machined (e.g., drilled) into the solid water equivalent material to hold a detector securely.

In some embodiments, the solid phantom comprises a base (e.g., a surface of the solid phantom that supports the weight of the solid phantom (e.g., opposing the force of gravity acting on the solid phantom)) and one or more external surfaces defining a volume.

In some embodiments, the solid phantom has a polygon (e.g., quadrilateral (e.g., rectangle))) shape when viewed from the top. However, the technology is not limited to solid phantoms having polygon, quadrilateral, and/or rectangle shapes when viewed from the top. Accordingly, embodiments provide solid phantoms having other shapes when viewed from the top, e.g., an “L” shape, a cross shape, a circular shape, etc.

In some embodiments, the solid phantom comprises a detector. In some embodiments, the solid phantom comprises a hole, and the hole comprises a detector. In some embodiments, the solid phantom comprises a number of holes, and one or more of the number of holes comprise(s) a detector. In some embodiments, the solid phantom comprises 1 to 10 holes (e.g., 1, 2, 3, 4, 5, 6, 7, 8, 9, or 10 holes), and each of a number of the 1 to 10 holes (e.g., 1, 2, 3, 4, 5, 6, 7, 8, 9, or 10 holes) comprises a detector. In some embodiments, a detector is removable from the solid phantom, e.g., for calibration, replacement, maintenance, and/or repair. In some embodiments, holes are precisely machined (e.g., drilled) into the solid water equivalent material to hold a detector securely. In some embodiments, a detector fits securely into a hole provided (e.g., by precise machining) in the solid water equivalent material of the solid phantom.

In some embodiments, the detector is structured to measure the energy, intensity, and/or dose of a radiation beam. In some embodiments, the detector is structured to measure one or more other characteristics of the radiation beam (e.g., shape, spectrum, wavelength, flux, photon count, etc.) In some embodiments, the detector comprises an ion chamber.

In some embodiments, the detector is moveable within the solid phantom. In some embodiments, the detector is removable from a first hole of the solid phantom and insertable into a second hole of the solid phantom. In some embodiments, the solid phantom comprises more than one detector (e.g., in some embodiments, the solid phantom comprises 2, 3, 4, 5, 6, 7, 8, or more detectors). In some embodiments, the solid phantom comprises a plurality of detectors and each detector is present in a hole of a plurality of holes of the solid phantom.

In some embodiments, the solid phantom comprises two detectors that are oriented at right angles to each other, e.g., so that a radiation beam is detected by both detectors as the solid phantom is rotated by 90°. In some embodiments, the solid phantom comprises two detectors that are oriented at an 180° angle to each other, e.g., so that a radiation beam is detected by both detectors as the solid phantom is rotated by 180°.

In some embodiments, the solid phantom comprises a single cylindrical detector that is oriented in a vertical direction along the rotation axis of the solid phantom (e.g., the solid phantom comprises a hole and the detector is present in the hole), e.g., so as to present a detection surface at a constant distance from the source (e.g., at a fixed location) along the axis of the radiation beam for all angles of rotation of the solid phantom. In this way, the distance between the radiation source and the detector remains constant as the solid phantom is rotated.

In some embodiments, the detector is movable within the solid phantom (e.g., the detector is removable from a first hole of the solid phantom and insertable into a second hole of the solid phantom) and the solid phantom defines a measurement area (or a measurement volume), e.g., because beam characteristics may be measured at one or more locations within the solid phantom.

In some embodiments, the phantom (e.g., a water phantom or a solid phantom) comprises a detector. In some embodiments, the detector comprises an ion chamber. In some embodiments, the detector is structured to detect megavoltage (e.g., treatment) x-ray beams. Without being limiting, exemplary detectors that find use in embodiments of the technology are, e.g., a FARMER type detector such as the EXRADIN A12 and EXRADIN A19 sold commercially by Standard Imaging; a 30010 or 30012 ionization chamber sold commercially by PTW; the FC65-G ionization chamber sold commercially by Iba Dosimetry; and the SNC15c, SNC350p, or SNC600c sold commercially by Sun Nuclear. Without being limiting, detectors (e.g., ionization chambers) are described in “Absorbed Dose Determination in External Beam Radiotherapy: An International Code of Practice for Dosimetry Based on Standards of Absorbed Dose to Water”, Technical Reports Series No. 398, International Atomic Energy Agency, Vienna, 2000, incorporated herein by reference, and particularly in Section 4.2.1 and in Tables 3 and 4.

### Methods

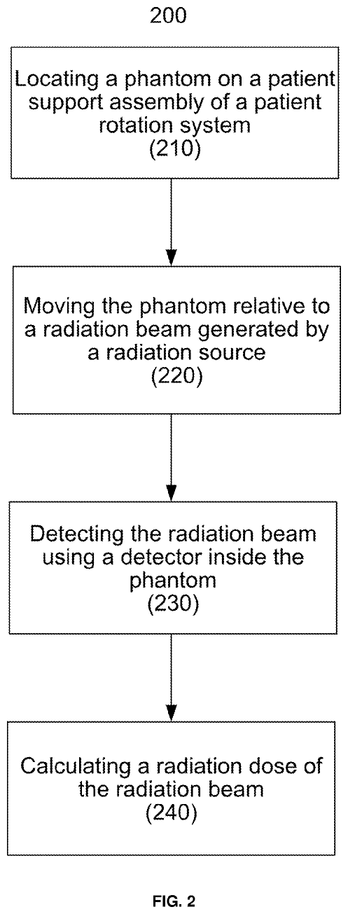

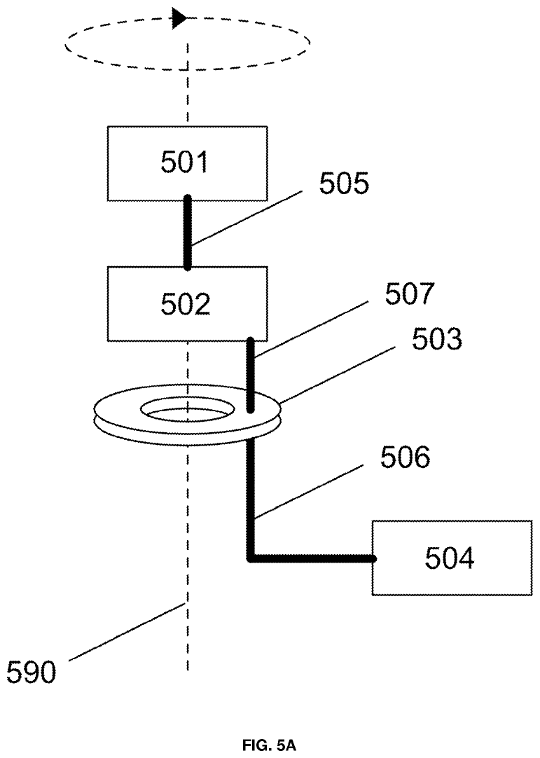

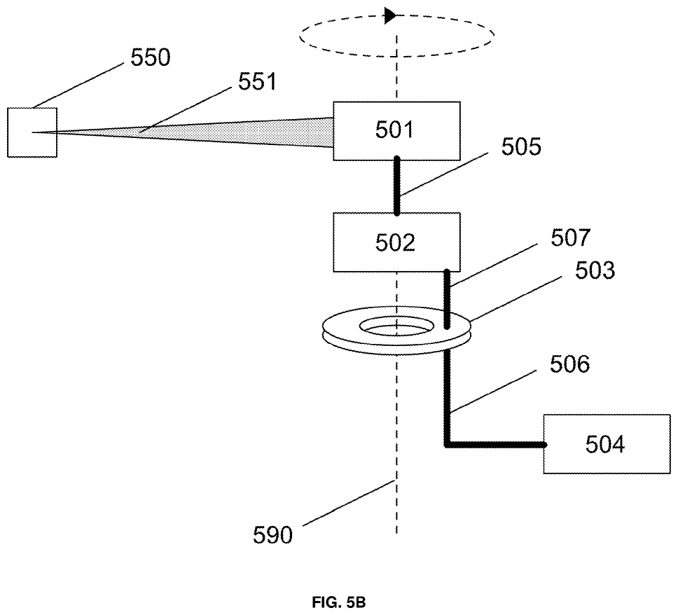

In some embodiments, e.g., as shown in FIG. 2, the technology provides methods for measuring a radiation dose provided by a medical radiation system comprising a patient rotation system and a radiation source (e.g., a static source). For example, in some embodiments, the method 200 comprises locating 210 a phantom (e.g., a water phantom comprising a tank, water, and a detector; or a solid phantom comprising a solid water equivalent material and a detector) on a patient support assembly of the patient rotation system. In some embodiments, the method comprises using a phantom system comprising a phantom (comprising a detector), an electrometer, and a slip ring, e.g., as shown in FIG. 5A and FIG. 5B. Thus, in some embodiments, methods comprise providing a phantom system comprising a phantom (comprising a detector), an electrometer, and a slip ring, e.g., as shown in FIG. 5A and FIG. 5B.

Further, in some embodiments, methods comprise moving 220 the phantom relative to a radiation beam generated by the radiation source. In some embodiments, methods comprise detecting 230 the radiation beam. In some embodiments, methods comprise calculating 240 the radiation dose of the radiation beam.

In some embodiments, the method 200 finds use in measuring the radiation dose of a medical radiation system comprising a patient rotation system adapted to rotate about a rotation axis. The radiotherapy system 100, e.g., as shown in FIG. 1, is an example of one such medical radiation system. However, embodiments of the method 200 are not limited for use with the radiotherapy system 100 described herein. Accordingly, embodiments provide that the method 200 finds use in measuring the radiation dose of any medical radiation system (e.g., any radiotherapy system and/or any medical imaging system).

In some embodiments, locating 210 the phantom (e.g., a water phantom comprising a tank, water, and a detector; or a solid phantom comprising a solid water equivalent material and a detector) on the patient support assembly comprises attaching or mounting the phantom to a component of the patient support assembly. In some embodiments, the phantom is attached to a seat member of the patient support assembly. In some embodiments, the phantom is mounted to an arm rest or to arm rests of the patient support assembly. For example, embodiments provide that the phantom may be mounted to one or more arm rests using an interface configured for holding quality assurance equipment. In some embodiments, the phantom is secured to any other part or component of the patient support assembly, e.g., embodiments provide that the phantom is secured to a part or component that is generally structured to support or to immobilize a patient. In some embodiments, a phantom system comprises the phantom (comprising a detector), an electrometer, and a slip ring, e.g., as shown in FIG. 5A and FIG. 5B.

In some embodiments, the phantom (e.g., a water phantom comprising a tank, water, and a detector; or a solid phantom comprising a solid water equivalent material and a detector) is located on the patient support assembly such that the rotation axis passes through the phantom and/or through an internal volume of the phantom. In some embodiments, a geometric center of the phantom is aligned with the rotation axis. In some embodiments, a geometric center of the phantom is aligned with the isocenter of the medical radiation system. In some embodiments, method 200 further comprises aligning the geometric center of the phantom with the rotation axis. In some embodiments, the phantom is supported by a seat member of the patient support assembly and the phantom is aligned using arm rests of the patient support assembly. That is, in some embodiments, a seat member of the patient support assembly are used to support the weight of the phantom, while arm rests of the patient support assembly are used to align the phantom.

In some embodiments, the phantom (e.g., a water phantom comprising a tank, water, and a detector: or a solid phantom comprising a solid water equivalent material and a detector) is located on a horizontal surface of the patient support assembly (e.g., on a seat member). In some embodiments, the rotation axis is a vertical axis (e.g., a substantially and/or effectively vertical axis) and the rotation axis extends along a vertical dimension of the phantom. In some embodiments, the rotation axis is perpendicular (e.g., substantially and/or effectively perpendicular) to the radiation beam. Therefore, in some embodiments, the radiation beam travels (e.g., substantially and/or effectively travels) along a horizontal plane that is orthogonal (e.g., substantially and/or effectively orthogonal) to the vertical rotation axis. In some embodiments, the phantom is disposed horizontally on the patient support assembly such that a central axis of the radiation beam is parallel (e.g., substantially and/or effectively parallel) to a base of the phantom. In some embodiments, the base of the phantom is defined as the surface of the tank that supports the weight of the phantom (e.g., opposing the force of gravity acting on the phantom).

In some embodiments, the phantom (e.g., a water phantom comprising a tank, water, and a detector; or a solid phantom comprising a solid water equivalent material and a detector) is located on the patient support assembly such that a sidewall or external surface of the phantom faces the radiation source and the radiation beam passes through the sidewall or external surface and enters the phantom. In some embodiments, the location and orientation of the phantom is such that the sidewall or external surface facing the radiation source is orthogonal to a central axis of the radiation beam.

In some embodiments, moving 220 the phantom (e.g., a water phantom comprising a tank, water, and a detector; or a solid phantom comprising a solid water equivalent material and a detector) comprises rotating the phantom, e.g., by rotating the patient rotation system about the rotation axis. In some embodiments, moving 220 the phantom comprises translating the phantom, e.g., by translating the patient support assembly relative to the patient rotation system. In some embodiments, moving 220 the phantom comprises moving the patient support assembly relative to the patient rotation system (e.g., without shifting the rotation axis of the patient rotation system). For example, in some embodiments, the patient support assembly is adapted for rotational movement (e.g., yaw, pitch, and/or roll) relative to a base of the patient rotation system. In some embodiments, moving the phantom comprises moving the patient rotation system relative to the fixed radiation source (e.g., without shifting the rotation axis of the patient rotation system). For example, in some embodiments, the patient rotation system is mounted to a translatable member (e.g., moveable in a vertical direction or in any other direction), thus allowing the whole patient rotation system to be translated in space. In some embodiments, rotating the phantom comprises also rotating the phantom, electrometer, and cable. In some embodiments, rotating the phantom comprises also rotating a first, phantom-side component of a slip ring. See FIG. 5A and FIG. 5B.

In some embodiments, the phantom (e.g., a water phantom comprising a tank, water, and a detector; or a solid phantom comprising a solid water equivalent material and a detector) is rotated in a continuous cycle. In some embodiments, the phantom is rotated in a discontinuous cycle. For example, in some embodiments, the phantom is rotated by 90°, after which detecting 230 the radiation beam and calculating 240 the radiation dose of the radiation beam are executed prior to resuming rotation of the phantom. In some embodiments, the phantom is rotated by any angle (e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350°) after which detecting 230 the radiation beam and calculating 240 the radiation dose of the radiation beam are executed prior to resuming rotation of the phantom through any angle (e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350°). In some embodiments, methods comprise waiting for a period of time after completing a step of rotating the phantom (e.g., a water phantom), e.g., after stopping the rotation and the phantom is stationary, to allow the liquid (e.g., water) to stop moving, e.g., to stabilize the liquid (e.g., water) and thus minimize and/or eliminate variation in the level of the liquid (e.g., water).

Alternatively, in some embodiments, the phantom is continuously rotated (e.g., without interrupting the rotating) while detecting 230 the radiation beam and/or calculating 240 the radiation dose of the radiation beam is/are executed. In some embodiments, methods comprise detecting 230 a radiation beam during moving 220 (e.g., rotating and/or translating) the phantom or detecting 230 a radiation beam within the relaxation time requirements of the liquid. Accordingly, in some embodiments, methods comprise providing and/or using a solid phantom. Embodiments of the solid phantom described herein provide advantages relative to water phantoms for detecting the radiation beam while the phantom is being rotated because the solid water equivalent material of the solid phantom remains at a constant level during rotation while the water level of a water phantom may vary due to acceleration of the phantom and water within the phantom tank during rotation.

In some embodiments, detecting 230 the radiation beam comprises detecting the radiation beam using a detector located within the phantom (e.g., a water phantom comprising a tank, water, and a detector; or a solid phantom comprising a solid water equivalent material and a detector). In some embodiments related to a water phantom, the detector is fully immersed in the liquid of the tank. In some embodiments related to a water phantom, the detector is partially immersed in the liquid of the tank. In some embodiments, detecting the radiation beam comprises measuring a dose of the radiation beam. In some embodiments, an energy (e.g., average energy) and/or an intensity of the radiation beam is/are calculated from one or more dose measurements.

Therefore, in some embodiments, the detector is structured to measure the dose of the radiation beam. In some embodiments, the detector is structured to measure one or more other characteristics of the radiation beam (e.g., dose profile).

In some embodiments, the detector is moveable within the phantom (e.g., a water phantom comprising a tank, water, and a detector; or a solid phantom comprising a solid water equivalent material and a detector). In some embodiments related to a water phantom, the detector is mounted to an adjustable arm that is fixed to the tank, such that the location and/or orientation of the detector may be adjusted using the adjustable arm. In some embodiments related to a solid phantom, the detector may be moved among a plurality of holes (e.g., removed from a first hole and inserted into a second hole). In some embodiments, detecting the radiation beam comprises positioning the detector to intercept the radiation beam by moving the phantom and/or by moving the detector within the phantom. A radiation beam spreads out in space as it travels (e.g., due to divergence and/or scatter); accordingly, in some embodiments, detecting the radiation beam comprises detecting the radiation beam at a plurality of locations within the phantom to detect multiple rays of the beam as the rays diverge.

In some embodiments, more than one detector is provided in the phantom to detect the radiation beam as the phantom is rotated (e.g., 2, 3, 4, 5, 6, 7, or 8 detectors are provided in the phantom to detect the radiation beam as the phantom is rotated). In some embodiments, two detectors are oriented at right angles to each other so that the radiation beam is detected as the phantom is rotated by 90°. In some embodiments, two detectors are oriented opposite each other so that the radiation beam is detected as the phantom is rotated by 180°. In some embodiments, a single cylindrical detector is oriented in a vertical direction along the rotation axis so as to present a detection surface at a constant distance from the source (e.g., at a fixed location) along the axis of the radiation beam for all angles of rotation of the phantom. In this way, the distance between the radiation source and the detector remains constant as the phantom is rotated.

In some embodiments, calculating 240 the radiation dose of the radiation beam comprises combining multiple measurements of the radiation beam from the detector(s). In some embodiments, calculating the radiation dose of the radiation beam comprises generating a spatial profile (e.g., a two-dimensional or a three-dimensional profile) of the intensity of the radiation beam within the phantom. In some embodiments, the detector is movable within the phantom and the phantom defines a measurement area (or a measurement volume), e.g., because beam characteristics may be measured at one or more locations within the phantom.

### Dose Scanning Systems and Methods

In some embodiments, e.g., as shown in FIG. 3, the technology provides systems for conducting a dose scan, e.g., a full three-dimensional (3D) dose scan. In some embodiments, the dose scan finds use in measuring a dose distribution, commissioning a linear accelerator (linac), and/or in performing quality assurance and/or maintenance processes for a medical radiation system. In some embodiments, a dose scan provides a characterization of the three-dimensional intensity profile of a radiation beam. Embodiments provide that the scan is conducted for a series of beam field sizes. In some embodiments, data obtained through these measurements are used to build a three-dimensional map of the radiation beam.

In some embodiments, systems comprise a phantom 310 (e.g., a water phantom comprising a tank, water, and a detector; or a solid phantom comprising a solid water equivalent material and a detector). In some embodiments, the phantom 310 is located on a patient support assembly of a patient rotation system structured to rotate about a rotation axis 320. In some embodiments, the phantom 310 is positioned such that a segment of rotation axis 320 is located within a sidewall or external surface of phantom 310. That is, in some embodiments, the sidewall or external surface of phantom 310 facing a radiation source 350 is parallel (e.g., substantially and/or effectively parallel) to rotation axis 320, which passes along the plane of the sidewall or external surface.

In the exemplary embodiment shown in FIG. 3, a first beam 330 and a second beam 340 are illustrated as being incident upon phantom 310 (e.g., a water phantom comprising a tank, water, and a detector; or a solid phantom comprising a solid water equivalent material and a detector). As an example, e.g., as shown in FIG. 3, the first beam 330 and the second beam 340 have different areas projected onto the phantom 310. For example, beam 330 may have dimensions of 10 cm by 10 cm, and beam 340 may have dimensions of 30 cm by 30 cm (e.g., corresponding to the dimensions of the semi-minor and semi-major axes of the ellipse defined by the area of the beam incident upon phantom 310). In this example, the larger sidewall or external surface of phantom 310 may be 100 cm long. The technology is not limited to a phantom 310 having a sidewall or external surface of 100 cm and/or beams having areas of 10 cm by 10 cm and 30 cm by 30 cm. Accordingly, the technology relates to beams and a phantom having any other length or area. In some embodiments, the phantom and the radiation source 350 are arranged such that the beam area is contained within the phantom. That is, embodiments provide that a dimension (e.g., height and/or width) of the area of the beam as projected onto the phantom does not exceed one or more dimensions (e.g., height and/or width) of the sidewall or external surface of the phantom onto which the beam is incident.

In some embodiments, the first beam 330 and the second beam 340 are generated asynchronously. In some embodiments, the first beam 330 and the second beam 340 are generated simultaneously (e.g., substantially and/or effectively simultaneously). In some embodiments, the first beam 330 and the second beam 340 are generated by the same source. In some embodiments, the first beam 330 and the second beam 340 are generated by different sources (e.g., the first beam 330 is generated by a first source and the second beam 340 is generated by a second source). In some embodiments, a beam shaping system is provided to adjust a size and/or shape (e.g., in one or two dimensions) of the radiation beam.

In some embodiments, systems comprise a detector (e.g., a detector of a water phantom or a solid phantom). In some embodiments, the detector is located inside phantom 310. In some embodiments, the detector is moved within the phantom to obtain a three-dimensional profile of the radiation beam. In some embodiments, the detector is moved along a vertical axis, a horizontal axis, and/or a depth axis to detect the beam intensity at multiple points within phantom 310. In some embodiments, the detector is translated along a vertical axis, a horizontal axis, and/or a depth axis to detect the beam intensity at multiple points within phantom 310. In some embodiments, the detector is rotated along a vertical axis, a horizontal axis, and/or a depth axis (e.g., providing a pitch, roll, and/or yaw rotation) to detect the beam intensity at multiple points within phantom 310.

In some embodiments, the patient support assembly is moved to obtain a three-dimensional profile of the radiation beam. In some embodiments, the patient support assembly is moved along a vertical axis, a horizontal axis, and/or a depth axis to detect the beam intensity at multiple points within phantom 310. In some embodiments, the patient support assembly is translated along a vertical axis, a horizontal axis, and/or a depth axis to detect the beam intensity at multiple points within phantom 310. In some embodiments, the patient support assembly is rotated along a vertical axis, a horizontal axis, and/or a depth axis (e.g., providing a pitch, roll, and/or yaw rotation) to detect the beam intensity at multiple points within phantom 310.

Some embodiments combine moving the detector in combination with moving the patient support assembly to detect the beam intensity in three-dimensional space. For example, in some embodiments, the detector is translated along and/or rotated around a vertical axis, a horizontal axis, and/or a depth axis and the patient support assembly is translated along and/or rotated around a vertical axis, a horizontal axis, and/or a depth axis. As an example, the detector may be moved in two dimensions (e.g., any two of a translation along a vertical axis, a translation along a horizontal axis, a translation along a depth axis, a rotation around a vertical axis, a rotation around a horizontal axis, and/or a rotation around a depth axis) while the patient support assembly is moved in a third dimension (e.g., any one of a translation along a vertical axis, a translation along a horizontal axis, a translation along a depth axis, a rotation around a vertical axis, a rotation around a horizontal axis, and/or a rotation around a depth axis); or, alternatively, the detector may be moved in one dimension (e.g., any one of a translation along a vertical axis, a translation along a horizontal axis, a translation along a depth axis, a rotation around a vertical axis, a rotation around a horizontal axis, and/or a rotation around a depth axis) while the patient support assembly is moved in two dimensions (e.g., any two of a translation along a vertical axis, a translation along a horizontal axis, a translation along a depth axis, a rotation around a vertical axis, a rotation around a horizontal axis, and/or a rotation around a depth axis).

### Tissue Phantom Ratio Measurement Systems and Methods

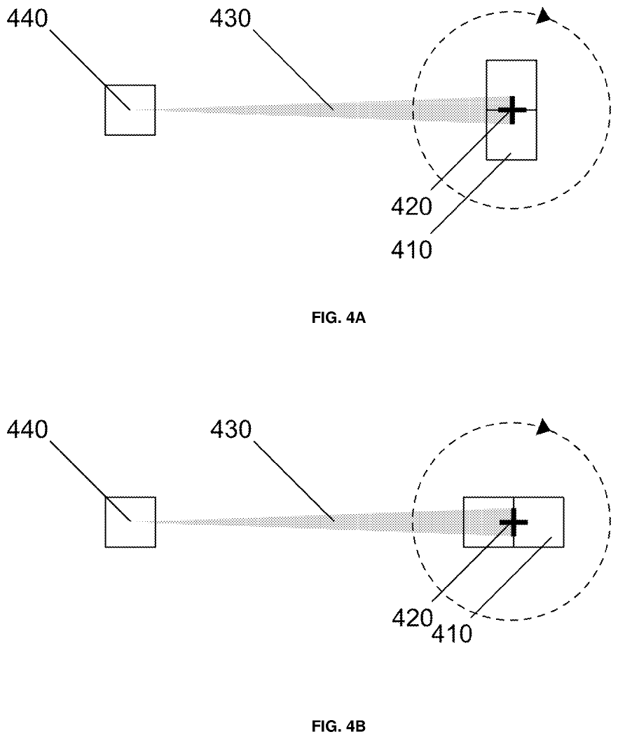

In some embodiments, e.g., as shown in FIG. 4A and FIG. 4B, the technology provides systems for conducting a tissue phantom ratio measurement. In some embodiments, a phantom 410 (e.g., a water phantom comprising a tank, water, and a detector; or a solid phantom comprising a solid water equivalent material and a detector) is located on a patient support assembly of a patient rotation system structured to rotate about a rotation axis 420. In some embodiments, phantom 410 is positioned such that rotation axis 420 intersects an inner volume of phantom 410 (e.g., rotation axis 420 passes through phantom 410). In some embodiments, a detector is placed within the phantom 410 on the rotation axis 420 to detect radiation beam 430. Accordingly, in some embodiments, the detector is positioned within the phantom at a location having a distance from the source 440 that is constant (e.g., substantially and/or effectively constant) as the phantom is rotated about the rotation axis.

FIG. 4A illustrates phantom 410 (e.g., a water phantom comprising a tank, water, and a detector; or a solid phantom comprising a solid water equivalent material and a detector) in a first orientation in which beam 430 reaches the detector after travelling a first distance inside phantom 410. FIG. 4B illustrates phantom 410 in a second orientation (e.g., rotated by 90° clockwise relative to the first orientation) in which beam 430 reaches the detector after travelling a second distance inside phantom 410, where the second distance is longer than the first distance. In both the first orientation of phantom 410 and the second orientation of phantom 410, a sidewall or external surface of phantom 410 facing the radiation source 440 is orthogonal to a central axis of beam 430. In some embodiments, the phantom is rotated by any other angle between the first orientation and second orientation (e.g., the phantom is rotated by 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or 350°).

In some embodiments, the beam 430 contacts the detector and the detector detects the beam 430. Accordingly, embodiments provide that the length of the propagation path of beam 430 is defined as the distance travelled by beam inside phantom 410 (e.g., a water phantom comprising a tank, water, and a detector; or a solid phantom comprising a solid water equivalent material and a detector). As used herein, the term “propagation path” refers to the straight-line distance between the sidewall or external surface of the phantom facing a radiation source 440 and a detector within the phantom. In some embodiments, e.g., as shown in FIG. 4A and FIG. 41B, the length of the propagation path of beam 430 inside phantom 410 in the first orientation (e.g., 10 cm) is different than the length of the propagation path of beam 430 inside phantom 410 in the second orientation (e.g., 20 cm). Embodiments of methods comprise measuring a dose and/or calculating a quality (e.g., energy, intensity, flux, attenuation, absorbed dose) of the beam using the phantom in the first orientation, measuring a dose and/or calculating the quality (e.g., energy, intensity, flux, attenuation, absorbed dose) of the beam using the phantom in the second orientation, and comparing the first measurement or calculated quality with the second measurement or calculated quality to calculate a tissue phantom ratio (TPR) characterizing the travel of the beam 430 through phantom 410. A TPR measurement, which may be a function of the energy or intensity of a beam, is an indicator of the quality of the beam.

In some embodiments, methods comprise calculating a TPR_{20,10}. In some embodiments, calculating a TPR_{20,10} comprises using embodiments of the systems and/or phantom 410 described herein, e.g., a phantom (e.g., a water phantom comprising a tank, water, and a detector; or a solid phantom comprising a solid water equivalent material and a detector) having dimensions and a detector located inside the phantom (e.g., as shown in FIG. 4A and FIG. 4B) such that the first propagation path length is 10 cm and the second propagation path length is 20 cm. In some embodiments, the ratio of the measurements at 20 cm and 10 cm provide a measure of radiation quality (e.g., the average energy of the beam).

The technology is not limited to calculating a TPR_{20,10} or to use of a phantom (e.g., a water phantom comprising a tank, water, and a detector; or a solid phantom comprising a solid water equivalent material and a detector) having dimensions and detector placement as described herein and shown in FIG. 4A and FIG. 4B. Accordingly, the technology provides that other propagation path lengths may be measured by changing the dimensions of phantom 410, by using different angles of rotation, or by changing the location of the detector inside phantom 410.

In some embodiments, the technology described provides methods, devices, and systems for measuring x-ray dose and/or calculating average beam energy or beam quality for an isocentric dose. In some embodiments, the technology described provides methods, devices, and systems for measuring x-ray beam quality and expressing the x-ray beam quality as a tissue phantom ratio (TPR). In some embodiments, measuring x-ray beam quality and expressing the x-ray beam quality as a tissue phantom ratio (TPR) (e.g., for an isocentric dose) comprises applying a translational and/or rotational offset of the patient rotation system to measure the beam at different propagation lengths (e.g., 10 and 20 cm). The rotational and/or translational movement of the phantom allows different measurements to be carried out without a technician or other operator having to access the medical radiation system or rearranging the measurement setup repeatedly. In some embodiments, the present method allows for efficient positioning of a measurement system (e.g., a phantom (e.g., a water phantom comprising a tank, water, and a detector; or a solid phantom comprising a solid water equivalent material and a detector)) for measuring a radiation dose of a medical radiation system. In some embodiments, a microprocessor (e.g., performing instructions provided by software) controls measurement of beam characteristics, movement (e.g., translation and/or rotation) of the phantom, and calculation of a TPR. In some embodiments, a microprocessor outputs the TPR value to a user. In some embodiments, a microprocessor outputs the TPR value to a user after an electrometer processes the signal and the processed charge signal and/or processed current signal from the ion chamber is transmitted through the slip ring to the user.

### Phantom Systems

In some embodiments, e.g., as shown in FIG. 5A and FIG. 5B, the technology provides a phantom system 500 for measuring a radiation dose. In some embodiments, phantom systems 500 comprise a phantom 501 (e.g., a water phantom or a solid phantom) comprising a detector, and further comprising an electrometer 502, a slip ring 503, and a microprocessor 504. In some embodiments, a microprocessor or computer comprises the electrometer. In some embodiments, the electrometer is a voltmeter and/or ammeter with a very high input impedance (e.g., at least approximately 10¹⁴ ohms) so that it resolves a very small signal (e.g., less that 1 pC and/or 1 nA). In some embodiments, the electrometer 502 is removable from the phantom system 500, e.g., for calibration, replacement, maintenance, and/or repair of the electrometer.

In some embodiments, the phantom 501 detector is in electric or electronic communication with the electrometer 502. In some embodiments, the phantom 501 detector is in electric or electronic communication with the electrometer 502 through a cable 505 connecting the detector and the electrometer 502. In some embodiments, the cable 505 connecting the detector and the electrometer 502 is a triaxial cable. In some embodiments, the electrometer 502 is in electric or electronic communication with a microprocessor 504 (e.g., a computer). In some embodiments, the electrometer 502 outputs a signal that is communicated to a microprocessor 504 (e.g., a computer). In some embodiments, the electrometer 502 outputs a signal that is communicated over a slip ring 503 to a microprocessor 504 (e.g., a computer). In some embodiments, the slip ring 503 comprises a first, phantom-side component that rotates with the phantom 501 and the electrometer 502; and comprises a second, user-side component that does not rotate with the phantom 501 and electrometer 502. The first, phantom-side component of the slip ring 503 and the second, user-side component of the slip ring 503 are in electric or electronic communication with each other. In some embodiments, the first, phantom-side component of the slip ring 503 is in electric or electronic communication with the electrometer 502 through cable 507; and the second, user-side component of the slip ring 503 is in electric or electronic communication with the microprocessor 504 through cable 506. In some embodiments, the system 500 further comprises an analog-to-digital converter (ADC) that converts the electrical (e.g., analog) signal produced by the electrometer 502 into a digital signal for communication (e.g., over the slip ring 503) to the microprocessor 504.

In some embodiments, methods comprise locating the phantom 501 (e.g., a water phantom comprising a tank, water, and a detector; or a solid phantom comprising a solid water equivalent material and a detector) of the phantom system 500 on a patient support assembly of the patient rotation system. In some embodiments, the electrometer 502 and cable 505 (e.g., triaxial cable) of the phantom system 500 are also located on a patient support assembly of the patient rotation system. In some embodiments, methods comprise rotating the patient rotation system around axis of rotation 590 so that the phantom 501, electrometer 502, and cable 505 (e.g., triaxial cable) of the phantom system 500 rotate around axis of rotation 590. The first, phantom-side component of the slip ring 503 also rotates around the axis of rotation 590 while remaining in electric or electronic communication with the second, user-side component of the slip ring 503. During rotation of the patient rotation system (and thus during rotation of phantom 501, electrometer 502, and cable 505 (e.g., triaxial cable)) around axis of rotation 590, the microprocessor 504 and cable 506 do not rotate around axis of rotation 590. The second, user-side component of the slip ring 503 also does not rotate around axis of rotation 590. See FIG. 5A.

In some embodiments, e.g., as shown in FIG. 5B, the technology provides a phantom system 500 for measuring a radiation dose provided by a beam 551 produced by a static source 550. In some embodiments, methods comprise rotating the patient rotation system around axis of rotation 590 so that the phantom 501, electrometer 502, and cable 505 (e.g., triaxial cable) of the system 500 rotate around axis of rotation 590; and producing a beam 551 from static source 550. Thus, in some embodiments, methods comprise contacting a detector of the phantom 501 with the beam 551 while the phantom 501 is rotating.

Accordingly, the phantom system 500 comprises a rotating subsystem comprising the phantom 501, electrometer 502, and cable 505 (e.g., triaxial cable); and the phantom system 500 comprises a non-rotating subsystem comprising the microprocessor 504. In some embodiments, the rotating subsystem comprises an ADC. In some embodiments, the rotating subsystem comprises a microprocessor or computer that comprises the electrometer. Thus, in some embodiments, data is carried from inside the rotating subsystem (e.g., from the phantom side of the system) to outside the rotating subsystem (to the user side) over the slip ring 503. In addition, the charges and/or currents output from the detector are sufficiently small that they are transmitted over a special cable (e.g., a triaxial cable) from the detector to the electrometer. Only the post-electrometer signal (e.g., analog charge or current measurement or digitized version thereof) is passed through the slip ring to the user side (e.g., outside a radiation bunker).

Although the disclosure herein refers to certain illustrated embodiments, it is to be understood that these embodiments are presented by way of example and not by way of limitation.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method of measuring a radiation dose provided by a medical radiation system comprising a patient rotation system and a radiation source, the patient rotation system being adapted to rotate about a rotation axis, the method comprising:
locating a phantom on a patient support assembly of the patient rotation system;
moving the phantom relative to a radiation beam generated by the radiation source;
detecting, using the detector, the radiation beam; and
calculating the radiation dose of the radiation beam.

2. The method of claim 1, wherein the phantom is located on the patient support assembly such that the rotation axis passes through the phantom.

3. The method of claim 1 or 2, wherein the phantom is located on the patient support assembly such that a sidewall or external surface of the phantom faces the radiation source and the radiation beam passes through the sidewall or external surface.

4. The method of claim 3, wherein the sidewall or external surface is orthogonal to a central axis of the radiation beam.

5. The method of claim 3 or 4, wherein the sidewall or external surface is transparent to the radiation beam.

6. The method of any one of claims 1 to 5, wherein moving the phantom comprises rotating the phantom by rotating the patient rotation system about the rotation axis.

7. The method of any one of claims 1 to 6, wherein moving the phantom comprises translating the phantom by translating the patient support assembly relative to the patient rotation system.

8. The method of any one of claims 1 to 7, wherein the detector is moveable within the phantom.

9. The method of claim 8, wherein detecting the radiation beam comprises positioning the detector to intercept the radiation beam by moving the phantom and moving the detector within the phantom.

10. The method of claim 8 or 9, wherein detecting the radiation beam comprises detecting the radiation beam at a plurality of locations within the phantom.

11. The method of any one of claims 1 to 10, wherein calculating the radiation dose of the radiation beam comprises generating a three-dimensional intensity profile of the radiation beam within the phantom.

12. The method of any one of claims 1 to 11, wherein the detector is located in line with the rotation axis.

13. The method of claim 12, wherein the calculated radiation dose is a first radiation dose obtained for a first orientation of the phantom, and wherein the method further comprises:
rotating the phantom to a second orientation, different from the first orientation, by rotating the patient rotation system about the rotation axis;
detecting, using the detector, the radiation beam for the second orientation;
calculating a second radiation dose of the radiation beam for the second orientation; and
obtaining a tissue phantom ratio by comparing the second radiation dose to the first radiation dose.

14. The method of claim 13, wherein the length of a first propagation path of the radiation beam inside the phantom for the first orientation is different from the length of a second propagation path of the radiation beam inside the phantom for the second orientation.

15. The method of claim 14, wherein the length of the first propagation path is 10 cm, and wherein the length of the second propagation path is 20 cm, such that the tissue phantom ratio is a TPR_{20,10} measurement.

16. The method of any one of claims 13 to 15, wherein a first sidewall or external surface of the phantom facing the radiation source in the first orientation is orthogonal to a central axis of the radiation beam.

17. The method claim 16, wherein a second sidewall or external surface of the phantom facing the radiation source in the second orientation is orthogonal to the central axis of the radiation beam.

18. The method of any one of claims 1 to 17, wherein the radiation source is one of an imaging radiation source or a therapeutical radiation source.

19. The method of any one of claims 1 to 18, wherein the rotation axis is perpendicular to the radiation beam.

20. The method of any one of claims 1 to 19, wherein the rotation axis is a vertical axis.

21. The method of any one of claims 1 to 20, wherein the phantom is securely attached to the patient support assembly.

22. The method of any one of claims 1 to 21, wherein the patient support assembly comprises an interface for attaching the phantom at a fixed position on the patient support assembly.

23. The method of any one of claims 1 to 22, wherein the phantom is mounted to a seat member of the patient support assembly.

24. The method of any one of claims 1 to 22, wherein the phantom is mounted to arm rests of the patient support assembly.

25. The method of any one of claims 1 to 24, wherein the phantom is located on a horizontal surface of the patient support assembly.

26. The method of any one of claims 1 to 25, wherein the phantom is disposed horizontally on the patient support assembly such that a central axis of the radiation beam is parallel to a base of the phantom.

27. The method of any one of claims 1 to 25, wherein the phantom is a water phantom comprising a tank, water, and a detector.

28. The method of any one of claims 1 to 25, wherein the phantom is a solid phantom comprising a solid water equivalent material and a detector.

29. A water phantom comprising:
a tank comprising a base, a first wall, and a second wall;
a detector located within the tank at a first distance from the first wall and located at a second distance from the second wall; and
water.

30. The water phantom of claim 29, wherein said first wall and/or said second wall comprises poly(methyl methacrylate).

31. The water phantom of claim 29, wherein said first wall is at an angle of 90° from said second wall.

32. The water phantom of claim 29, wherein said detector has a cylindrical shape.

33. The water phantom of claim 29, wherein said detector has a first detection face parallel with said first wall and a second detection face parallel with said second wall.

34. The water phantom of claim 29, wherein said first distance is 10 cm and said second distance is 20 cm.

35. The water phantom of claim 29, further comprising a component structured to attach the water phantom to a patient support assembly.

36. The water phantom of claim 29, wherein said detector is located at an axis of rotation of said water phantom.

37. The water phantom of claim 29, further comprising a movable arm operatively engaged with said detector.

38. A solid phantom comprising:
a solid water equivalent material comprising a first external surface and a second external surface; and
a detector located within the solid water equivalent material at a first distance from a first external surface and located at a second distance from the second external surface.

39. The solid phantom of claim 38, wherein said first external surface is at an angle of 90° from said second external surface.

40. The solid phantom of claim 38, wherein said detector has a cylindrical shape.

41. The solid phantom of claim 38, wherein said detector has a first detection face parallel with said first external surface and a second detection face parallel with said second external surface.

42. The solid phantom of claim 38, wherein said first distance is 10 cm and said second distance is 20 cm.

43. The solid phantom of claim 38, further comprising a component structured to attach the solid phantom to a patient support assembly.

44. The solid phantom of claim 38, wherein said detector is located at an axis of rotation of said solid phantom.

45. The solid phantom of claim 38, wherein said solid water equivalent material comprises a hole and the detector is located in the hole.

46. A system comprising:
a medical radiation system; and
a water phantom comprising:
a tank comprising a base, a first wall, and a second wall;
a detector located within the tank at a first distance from the first wall and located at a second distance from the second wall; and
water.

47. A system comprising:
a medical radiation system; and
a solid phantom comprising:
a solid water equivalent material comprising a first external surface and a second external surface; and
a detector located within the solid water equivalent material at a first distance from a first external surface and located at a second distance from the second external surface.

48. The system of any one of claim 46 or 47, wherein said medical radiation system comprises an x-ray source.

49. The system of any one of claim 46 or 47, further comprising a patient support assembly.

50. The system of claim 49, wherein said patient support assembly comprises an interface structured to accept said water phantom or said solid phantom.

51. The system of claim 49, wherein said patient support assembly is structured to operably engage said water phantom or said solid phantom.

52. The system of claim 49, wherein said patient support assembly is structured to move said water phantom or said solid phantom.

53. The system of claim 49, wherein said patient support assembly is structured to rotate said water phantom or said solid phantom.

54. The system of any one of claim 46 or 47, further comprising a beam.

55. The system of any one of claim 46 or 47, further comprising a software component comprising instructions for rotating said water phantom or said solid phantom.

56. The system of any one of claim 46 or 47, further comprising a software component comprising instructions for activating the source to produce a beam.

57. The system of any one of claim 46 or 47, further comprising a software component comprising instructions for receiving data from said detector and calculating a tissue phantom ratio using said data.

58. The system of claim 57, wherein said tissue phantom ratio is a TPR_{20,10}.

59. The system of claim 46, wherein said first wall and/or said second wall comprises poly(methyl methacrylate).

60. The system of any one of claim 46 or 47, wherein said first wall or said first external face is at an angle of 90° from said second wall or said second external face.

61. The system of any one of claim 46 or 47, wherein said detector has a cylindrical shape.

62. The system of any one of claim 46 or 47, wherein said detector has a first detection face parallel with said first wall or said first external face and a second detection face parallel with said second wall or said second external face.

63. The system of any one of claim 46 or 47, wherein said first distance is 10 cm and said second distance is 20 cm.

64. A phantom system for measuring a radiation dose, said system comprising:
a phantom comprising a detector;
a slip ring;
a microprocessor; and
an electrometer in electronic or electric communication with the detector through a cable and in electronic or electric communication with the microprocessor through the slip ring.

65. The phantom system of claim 64, wherein the phantom is a water phantom.

66. The phantom system of claim 64, wherein the phantom is a solid phantom.

67. The phantom system of claim 64, wherein the cable is a triaxial cable.

68. The phantom system of claim 64, wherein a computer comprises the electrometer.

69. The phantom system of claim 64, further comprising an analog-to-digital converter in electric communication with the electrometer.

70. The phantom system of claim 64, comprising a rotating subsystem comprising the phantom and electrometer.

71. The phantom system of claim 64, comprising a non-rotating subsystem comprising the microprocessor.

72. A method of measuring a radiation dose provided by a medical radiation system comprising a patient rotation system and a radiation source, the patient rotation system being adapted to rotate about a rotation axis, the method comprising:
locating a phantom of a phantom system on a patient support assembly of the patient rotation system;
moving the phantom relative to a radiation beam generated by the radiation source;
detecting, using the detector, the radiation beam;
producing, by an electrometer, an electrical signal characterizing the radiation beam;
communicating the electrical signal from the electrometer through a slip ring to a microprocessor; and
calculating the radiation dose of the radiation beam using the signal.

73. The method of claim 72, wherein the phantom is located on the patient support assembly such that the rotation axis passes through the phantom.

74. The method of claim 72, wherein moving the phantom comprises rotating the phantom by rotating the patient rotation system about the rotation axis.

75. The method of claim 72, wherein detecting the radiation beam comprises detecting the radiation beam at a plurality of locations within the phantom.

76. The method of claim 72, wherein calculating the radiation dose of the radiation beam comprises generating a three-dimensional intensity profile of the radiation beam within the phantom.

77. The method of claim 72, wherein the detector is located in line with the rotation axis.

78. The method of claim 72, wherein the calculated radiation dose is a first radiation dose obtained for a first orientation of the phantom, and wherein the method further comprises:
rotating the phantom to a second orientation, different from the first orientation, by rotating the patient rotation system about the rotation axis;
detecting, using the detector, the radiation beam for the second orientation;
producing, by the electrometer, a second electrical signal characterizing the radiation beam for the second orientation;
communicating the second electrical signal from the electrometer through the slip ring to a microprocessor;
calculating a second radiation dose of the radiation beam for the second orientation; and
obtaining a tissue phantom ratio by comparing the second radiation dose to the first radiation dose.

79. The method of claim 78, wherein the length of a first propagation path of the radiation beam inside the phantom for the first orientation is different from the length of a second propagation path of the radiation beam inside the phantom for the second orientation.

80. The method of claim 78, wherein the length of the first propagation path is 10 cm, and wherein the length of the second propagation path is 20 cm, such that the tissue phantom ratio is a TPR_{20,10} measurement.

81. The method of claim 72, wherein a first sidewall or external surface of the phantom facing the radiation source in the first orientation is orthogonal to a central axis of the radiation beam.

82. The method claim 72, wherein a second sidewall or external surface of the phantom facing the radiation source in the second orientation is orthogonal to the central axis of the radiation beam.

83. The method of claim 72, wherein the radiation source is one of an imaging radiation source or a therapeutical radiation source.

84. The method of claim 72, wherein the rotation axis is perpendicular to the radiation beam.

85. The method of claim 72, wherein the rotation axis is a vertical axis.

86. The method of claim 72, wherein the phantom is securely attached to the patient support assembly.

87. The method of claim 72, wherein the patient support assembly comprises an interface for attaching the phantom at a fixed position on the patient support assembly.

88. The method of claim 72, wherein the phantom is mounted to a seat member of the patient support assembly.

89. The method of claim 72, wherein the phantom is mounted to arm rests of the patient support assembly.

90. The method of claim 72, wherein the phantom is located on a horizontal surface of the patient support assembly.

91. The method of claim 72, wherein the phantom is disposed horizontally on the patient support assembly such that a central axis of the radiation beam is parallel to a base of the phantom.

92. The method of claim 72, wherein the phantom is a water phantom comprising a tank, water, and a detector.

93. The method of claim 72, wherein the phantom is a solid phantom comprising a solid water equivalent material and a detector.
